(19) United States
(12) Patent Application Publication
(10) Pub. No.: US 2025/0281655 A1
(43) Pub. Date: Sep. 11, 2025

(54) SYSTEMS AND METHODS FOR INFECTION CONTROL IN MEDICAL IMPLANTS AND BIOLOGICAL CONDUITS USING ELECTROPORATION

(71) Applicant: Ablation Innovations, LLC, Brentwood, TN (US)

(72) Inventors: Daniel Walter Kaiser, Nashville, TN (US); Margaret Rox, Brentwood, TN (US); Clayton Allen Kaiser, Nashville, TN (US)

(21) Appl. No.: 19/071,649

(22) Filed: Mar. 5, 2025

Related U.S. Application Data
(60) Provisional application No. 63/561,707, filed on Mar. 5, 2024.

(51) Int. Cl. A61L 2/03; A61L 2/24; A61N 1/05; A61N 1/36; G16H 40/63

(52) U.S. Cl. CPC A61L 2/03; A61L 2/24; A61N 1/05; A61N 1/3603; G16H 40/63; A61L 2202/14; A61L 2202/24

(57) ABSTRACT

Systems and methods for infection prevention and treatment in biological conduits using electroporation or pulsed field ablation (PFA). The system includes an elongate member, such as a dialysis catheter, vascular access device, or peritoneal dialysis conduit, with at least one electrode positioned along, within, or around the member. A control unit applies pulsed electric fields to inhibit microbial colonization, disrupt bacterial biofilms, and alter permeability of bacterial or endothelial cells to enhance sterility or fluid exchange. The system may use real-time impedance monitoring, microbial detection, or fluid composition analysis to adjust electroporation parameters. Electroporation may synchronize with dialysis cycles or antimicrobial lock solutions. The method includes automated, clinician-controlled, or sensor-triggered treatments to reduce infections and improve device longevity. The system provides an alternative or augment to antibiotics, reducing the risk of drug-resistant infections while improving the longevity and safety of medical implants.

### RELATED APPLICATION DATA

The present application claims benefit of co-pending U.S. provisional application Ser. No. 63/561,707, filed Mar. 5, 2024, the entire disclosure of which is expressly incorporated by reference herein.

### TECHNICAL FIELD

The present application relates to medical device and, more particularly, to systems and methods for infection prevention, treatment, and control in implantable medical devices, particularly those associated with biological conduits such as dialysis catheters, vascular grafts, peritoneal dialysis systems, and other fluid exchange pathways. The disclosed systems utilize electroporation or pulsed field ablation (PFA) to deliver controlled electrical pulses that selectively disrupt bacterial biofilms, reduce microbial colonization, and, in some cases, enhance solute permeability for improved dialysis efficiency.

### BACKGROUND

Congestive heart failure, chronic infections, and other long-term medical conditions often require continuous or frequent administration of therapeutic agents, such as diuretics, antibiotics, or other pharmacological compounds. Traditionally, patients may rely on oral medications, repeated intravenous (IV) injections, or transcutaneous drug delivery systems that are inconvenient, inconsistent, or susceptible to infections and complications. Patients with chronic conditions often experience difficulties in maintaining stable drug levels in the body, potentially leading to suboptimal treatment outcomes. Moreover, continuous access routes, such as indwelling catheters or external infusion pumps, are associated with an elevated risk of infection due to microorganisms colonizing the catheter entry sites, fluid pathways, or external components.

In parallel, patients who require cardiac support devices, such as pacemakers or implantable cardioverter defibrillators (ICDs), benefit from increasingly sophisticated implantable electronics that manage cardiac rhythm disorders. This integration of drug delivery and cardiac support offers a powerful way to treat comorbidities in a single device platform. Yet, current solutions to integrate both cardiological and pharmacological support remain limited. Additionally, the implantation of multiple devices in a single patient often results in complex surgical procedures, greater risk of infection, and mechanical complexity. Often when a pacing or ICD lead or device becomes infected, extraction is needed to fully eradicate the infection. Thus, it is desirable to incorporate infection prevention and control directly into these devices to mitigate high cost, high risk extractions and management of infection.

Peripheral Intravenous Catheters (PICCs), peripheral IVs, Foley catheters, and other similar medical devices are vital in healthcare for administering medications and fluids, and for patient monitoring. However, these devices pose a significant risk for infections, primarily due to their invasive nature and prolonged use. PICCs, dialysis ports, and Peripheral IVs: These catheters, inserted into veins for intravenous treatments, can be a conduit for pathogens to enter the bloodstream, leading to infections like bacteremia or sepsis. The risk is exacerbated by the duration of catheter use and the frequency of access. Foley Catheters: Used for urinary drainage, Foley catheters are associated with urinary tract infections (UTIs), the most common type of hospital-acquired infection. The risk increases with prolonged use and can lead to complications like bladder and kidney infections. The pathogens involved in these infections are often resistant to multiple drugs, making treatment challenging. Preventative measures are crucial, but there remains a need for innovative solutions to mitigate these risks.

Endocarditis and Vulnerability in Patients with Heart Valves: Endocarditis, an infection of the heart valves, is a severe condition that can be life-threatening. Patients with newly implanted heart valves, whether mechanical or biological, are at a heightened risk for endocarditis. Pathogenesis: The presence of foreign material in the heart can serve as a nidus for bacterial colonization, especially during the early postoperative period when the valve is not yet fully endothelialized. Endocarditis can lead to severe complications, including heart failure, systemic embolism, and the need for repeat heart surgery. The morbidity and mortality associated with valve-related endocarditis remain significant. Due to associated risks, these patients may be too sick for the surgery needed to treat bacterial infections. Given the high stakes for patients with new heart valves, preventing infections or alternative methods of treating them, particularly bloodstream infections originating from catheters, is of paramount importance.

Left Ventricular Assist Devices (LVADs): LVADS are critical life-sustaining devices for patients with advanced heart failure, serving either as a bridge to transplant or as destination therapy. Despite their life-saving potential, LVADs carry a significant risk of complications, with driveline infections being one of the most severe and frequent issues. Infection rates associated with LVAD drivelines are reported to be as high as 25% per year, leading to serious morbidity, hospital readmissions, and increased mortality. The current approaches to mitigating driveline infections involve systemic antibiotic prophylaxis, local antiseptic measures, and in some cases, surgical interventions. However, these strategies have not sufficiently eliminated the persistent risk of infection, underscoring the need for novel, more effective solutions.

Thus, there is a need for an integrated implantable medical device that can reliably and safely store and deliver multiple medications over an extended period, provide or coordinate with cardiac support functionalities, and minimize the risk of device-related infection. A particular challenge involves preventing or mitigating infection risk associated with implantable drug reservoirs and their refill ports. Conventional mechanical or chemical sterilization strategies are complex and not easily maintained within the patient's body over long durations. Electroporation or pulsed field ablation (PFA) techniques could offer a way to selectively disrupt bacterial cell membranes at or near the device's port and fluid pathways, reducing infection risk during periodic refills.

Existing infection control solutions for implantable medical devices have traditionally relied on chemical-based methods, such as antimicrobial coatings or electrochemical disinfection. While these techniques have demonstrated effectiveness in reducing initial bacterial colonization, they often suffer from limitations, including degradation over time and dependency on patient-specific chemical environments. The present electroporation system overcomes these challenges by providing a non-chemical, electricity-driven solution that selectively disrupts bacterial cell membranes without requiring replenishable coatings or external chemical agents.

Prior methods utilizing antimicrobial coatings introduce challenges in maintaining long-term efficacy, as these coatings gradually wear off or leach into the surrounding environment, reducing their protective properties. Furthermore, biofilm formation on catheter surfaces often diminishes the penetration capability of chemical treatments, necessitating frequent reapplications or device replacements. In contrast, the electroporation approach presented herein provides consistent bacterial elimination, leveraging pulsed electrical fields to directly disrupt microbial integrity, independent of chemical interactions.

Other conventional approaches, such as electrochemical disinfection, have attempted to use applied voltage to generate antimicrobial agents from endogenous compounds. However, these approaches rely on the availability of specific biochemical conditions within the patient's body, leading to variability in effectiveness. The present electroporation-based system circumvents this limitation by delivering controlled electrical pulses that mechanically and non-thermally disrupt bacterial membranes, ensuring a consistent and reliable sterilization effect regardless of environmental variability.

Electroporation and pulsed field ablation (PFA) have been widely explored in other medical applications, including gene therapy, tumor ablation, and cardiac treatments. Electroporation has been extensively utilized for targeted drug and gene delivery, enhancing cellular uptake without the need for viral vectors. Similarly, PFA has demonstrated efficacy in cardiac electrophysiology by selectively ablating tissue while preserving surrounding structures. These existing applications highlight the precision and adaptability of electroporation-based technologies, reinforcing their potential for safe and effective bacterial eradication in implantable medical devices.

Therefore, improved devices for treating congestive heart failure, chronic infections, and other long-term medical conditions would be useful.

### SUMMARY

The present disclosure provides systems and methods for infection prevention, treatment, and control in implantable medical devices, particularly those associated with biological conduits such as dialysis catheters, vascular grafts, peritoneal dialysis systems, and other fluid exchange pathways. The disclosed systems utilize electroporation or pulsed field ablation (PFA) to deliver controlled electrical pulses that selectively disrupt bacterial biofilms, reduce microbial colonization, and, in some cases, enhance solute permeability for improved dialysis efficiency.

Unlike prior electroporation-based systems that require an external ablation device to be inserted into the patient, in one example, the system includes both fully implantable and externally powered variations. In fully implantable configurations, the system operates autonomously within the body, detecting bacterial presence and applying targeted electroporation pulses without requiring external intervention. Alternatively, in some implementations, power may be applied externally through wireless energy transfer or a connected external power source, allowing for controlled electroporation treatment when needed.

This flexible design significantly improves patient comfort, reduces infection risks associated with repeated device insertion, and enhances overall treatment efficacy. By eliminating reliance on antimicrobial coatings or chemically reactive surfaces, the electroporation system offers an advanced infection control strategy that enhances patient safety, device longevity, and clinical efficiency. Its ability to integrate real-time sensing and adaptive energy delivery further ensures that bacterial eradication occurs precisely where needed, distinguishing it from prior solutions and establishing a new standard for infection prevention in implanted medical devices.

By eliminating reliance on antimicrobial coatings or chemically reactive surfaces, the electroporation system offers an advanced infection control strategy that enhances patient safety, device longevity, and clinical efficiency. Its ability to integrate real-time sensing and adaptive energy delivery further ensures that bacterial eradication occurs precisely where needed, distinguishing it from prior solutions and establishing a new standard for infection prevention in implanted medical devices.

One example includes a dialysis catheter system incorporating electroporation electrodes along its length or near its insertion site. The electrodes may be positioned within the catheter lumen, on its outer surface, or embedded in a deployable sheath that expands to conform to the conduit's walls. Electroporation pulses can be applied before, during, or after dialysis treatment cycles to prevent bloodstream infections, mitigate biofilm formation, and ensure catheter sterility over long-term use.

In some examples, electroporation is integrated into peritoneal dialysis catheters to prevent peritonitis and enhance the transport of solutes across the peritoneal membrane. Pulsed electric fields may transiently increase cell membrane permeability, improving the diffusion of dialysate, waste products, or therapeutic agents. The system may further incorporate adaptive impedance sensing or real-time bacterial detection to dynamically adjust electroporation parameters based on microbial load or tissue response.

For vascular grafts, hemodialysis shunts, or arteriovenous (AV) fistulas, electroporation may be applied to prevent biofilm accumulation, reduce thrombosis risk, and modulate endothelial function. In some examples, a flexible electrode mesh may be deployed within the graft lumen or along its external surface, applying targeted pulsed fields without disrupting normal blood flow.

Power for the electroporation system may be provided by implantable batteries, external energy sources, or wirelessly rechargeable units. In certain designs, energy may be harvested from fluid flow within the conduit to sustain low-energy electroporation pulses over extended periods.

In addition to infection control, electroporation may be used to enhance drug delivery in biologic conduits, particularly in conjunction with antibiotic lock solutions for dialysis ports. By increasing bacterial membrane permeability, electroporation may improve antimicrobial efficacy and reduce antibiotic resistance development.

Further examples include a pacemaker system adapted to deliver pulsed field ablation (PFA) or electroporation energy along pacemaker leads to reduce or eliminate biofilms that cause infection of implanted cardiac devices. The system employs electrodes that are printed, embedded, or otherwise integrated along the outer surface of the pacemaker leads, enabling localized electroporation of microbial cells. By periodically or on-demand applying electrical pulses, the system disrupts bacterial, fungal, or other pathogenic biofilms, thus decreasing the likelihood of systemic infection and potentially reducing or obviating the need for device extraction.

In one example, the pacemaker generator includes or interfaces with a PFA energy port-either directly accessible via needle insertion or via a noncontact inductive coupling mechanism. In this manner, an external or built-in energy source can deliver electroporation pulses to the implanted leads when an infection is suspected or confirmed. Additionally, antibiotics or antimicrobial agents administered concurrently may exhibit enhanced efficacy due to the increased permeability of microbial cell membranes caused by PFA.

The present application further provides an implantable medical device configured to store and deliver therapeutic agents internally while optionally providing or integrating with cardiac rhythm management functionalities. The device includes one or more drug-holding pouches or reservoirs, each capable of containing a distinct therapeutic agent, such as a diuretic, antibiotic, antiviral, anticoagulant, chemotherapeutic, or other medications beneficial for long-term management of chronic diseases. These pouches may be located transdermally in the patient's chest area, such as situated between subcutaneous fat layers and muscular structures, or within other suitable anatomical locations, including the abdominal cavity, to facilitate easy access and stable implantation.

The device includes a refillable port accessible through a minimally invasive percutaneous approach. This port allows healthcare providers to replenish drug supplies periodically without requiring major surgery. Integrated with the port or adjacent fluid pathways is an electroporation or pulsed field ablation (PFA) assembly designed to apply carefully controlled electrical fields to destroy or disrupt bacterial cell membranes, thereby reducing the risk of infection. The device further houses a processor or control unit to regulate drug release timing, dosage, and coordination with other implantable components, such as pacemaker leads, defibrillator leads, or sensors. In certain examples, the device can integrate fully with existing cardiac devices or include its own lead system to deliver electrical impulses to the heart.

In addition to delivering drugs and offering infection control measures, the device may incorporate physiological sensors to monitor patient status, including heart rate, blood pressure, fluid overload indicators, or biochemical markers. The processor can use this data to optimize drug delivery algorithms, ensuring that patients receive medication in a personalized, timely manner.

The system's processor or controller unit, enclosed within the device's housing, orchestrates multiple functions, including drug dosing algorithms, elective sterilization cycles, integration with cardiac pacing or defibrillation leads, and interaction with physiological sensors. The sensors can monitor parameters like heart rate, fluid status, pressure, biochemical markers, and potentially microbial load, adjusting therapy in real-time. Additional functionalities, such as wireless data transfer and remote programming, allow clinicians to tailor treatment regimens, upload new dosing protocols, and monitor device performance without invasive intervention.

The present application further provides systems and methods for mitigating catheter failure due to inflammatory and immune system responses commonly observed in indwelling vascular catheters. Foreign body response (FBR) plays a critical role in long-term catheter dysfunction, leading to macrophage recruitment, fibroblast proliferation, and fibrotic encapsulation. Over time, this process can cause stenosis, occlusion, or loss of catheter function. Additionally, biofilm formation and infection-triggered inflammation exacerbate immune activation, leading to sustained neutrophil recruitment, cytokine release (e.g., IL-6, TNF-α), and platelet aggregation, further increasing the risk of thrombosis and catheter blockage.

To counteract these challenges, the disclosed systems may incorporate electroporation or pulsed field ablation (PFA) technology to modulate immune response, disrupt biofilm formation, and prevent inflammatory cytokine cascades. Electroporation pulses may be applied in a targeted manner to break bacterial biofilms, while simultaneously delivering controlled energy profiles to reduce fibroblast overactivity, suppress excessive macrophage activation, and prevent thrombosis without damaging healthy endothelial cells.

In some examples, electroporation-enabled catheters may be designed with adaptive impedance monitoring, allowing real-time detection of inflammatory changes, clot formation, or fibrotic encapsulation. The system may automatically adjust electroporation pulse parameters or synchronize with anti-inflammatory drug coatings (e.g., dexamethasone-eluting catheters) to actively reduce immune activation and prolong catheter function.

By integrating electroporation-based immune modulation, real-time sensing, and biofilm disruption, the disclosed systems provide a comprehensive strategy for extending catheter longevity, reducing infection rates, and preventing immune-mediated catheter failure.

The present application additionally provides systems and methods for the prevention and/or treatment of bacterial infection that include medical catheters configured to perform electroporation and or pulsed-field ablation (PFA). Electroporation is a technique that applies electric fields to create temporary pores in cell membranes, primarily used for delivering drugs or genes into cells. This system utilizes the concept of electroporation to disrupt bacterial cells and prevent infection in and around the catheter site, or in other areas of the vasculature or the body that can be accessed by a catheter.

Electroporation and Bacterial Cell Disruption: Electroporation, the process of applying electric fields to create temporary pores in cell membranes, is a promising avenue for combating bacterial infections. By applying controlled electric pulses, electroporation can disrupt the integrity of bacterial cell membranes. This disruption leads to the loss of cellular contents and eventually cell death, effectively reducing bacterial load. Unlike antibiotics, electroporation does not rely on chemical agents, reducing the risk of developing drug-resistant strains. When correctly calibrated, it can be targeted to affect only bacterial cells without causing harm to human cells. Electroporation presents an innovative method to sterilize medical devices like catheters, potentially reducing the incidence of device-related infections.

The systems and methods herein may include a device sized to be inserted into the body that includes electrodes or other elements for delivering electric fields at specific parameters intended to infiltrate and damage bacterial cells without disrupting healthy tissue. In accordance with one example, electrodes may be embedded in catheter walls. This design integrates electrodes directly into the walls of the catheter. The electrodes are arranged in a pattern that ensures uniform distribution of the electric field along the catheter's surface. This configuration is ideal for long-term catheters like PICCs and Foley catheters.

In another example, electrodes may wrap externally around the catheter or other insertion device. For example, a flexible, conductive material wrap may be fitted around the external surface of the catheter. This wrap can be activated to deliver electroporation pulses. This design allows for retrofitting existing catheters with electroporation capabilities. Another example may include intermittent electrode rings. A series of ring-shaped electrodes are spaced along the length of the catheter. These rings can be individually controlled to target specific areas along the catheter, providing localized electroporation.

These or other examples herein may include a nanoelectrode coating. The catheter surface is coated with a layer of nanoscale electrodes. These electrodes can generate a finely tuned electric field, ideal for precise electroporation, minimizing damage to surrounding tissues.

Optionally, the systems and methods herein may include a conductive wire insert that can deliver electric pulses. For example, the insert may include a conductive wire, running through the catheter's lumen, that serves as an electrode. When activated, the conductive wire creates an electric field that permeates the catheter's walls, providing a uniform electroporation effect. Optionally, this or other examples may include an expandable electrode mesh. In catheters placed in larger vessels or lumens, an expandable mesh made of conductive material can be employed. This mesh can expand to contact the catheter's interior surface, ensuring comprehensive coverage for electroporation. In another example, the system and/or methods may include capacitive coupling electrodes. Electrodes placed on the outer surface of the catheter utilize capacitive coupling to create an electric field without direct contact with the body fluid or tissue, offering a non-invasive approach.

Each of these configurations is designed to maximize the efficacy of the electroporation process while ensuring patient safety and comfort. The system's control unit can select and vary the electroporation parameters, including waveform, intensity, and duration, to adapt to different clinical scenarios and catheter types. The control unit may optionally be connected and disconnected as needed to control the electroporation parameters.

The proposed electroporation systems and methods are intended to be versatile, allowing integration with various catheter designs to address a wide range of medical needs. In accordance with one example, the catheter or electroporation delivery apparatus may be designed to be steerable. As such, the apparatus may include one or more mechanisms allowing for directional control, making the apparatus ideal for navigating through complex vascular or organ pathways. Embedding electrodes in steerable catheters or wires can provide targeted electroporation in hard-to-reach areas. In this or other examples, the catheter may include an expandable tip, such as a catheter with a tip that can expand upon deployment, increasing surface contact with tissue. Electrodes embedded in these expandable tips can deliver electroporation pulses more effectively in larger vessels or cavities.

In accordance with another example, the catheter or delivery device may include multiple lumens. These catheters have multiple channels (lumens) for different functions (e.g., fluid delivery, monitoring). Electrodes can be integrated into one or more lumens for simultaneous electroporation and other medical procedures. Optionally, the device (such as a wire, needle, tube, or otherwise) may be delivered through a lumen of a multi-lumen catheter to apply targeted pulses. In another example, the delivery system may be a needle catheter. Combining the features of a needle and a catheter, these are used for precise access to specific tissues. Miniaturized electrodes can be placed along the needle shaft for localized electroporation at the target site. In these or any other examples herein, the electroporation delivery system may include a balloon on the distal end or at any point along the length of the catheter, wire, etc. Widely used in procedures like angioplasty, similar catheters have a balloon at the tip. Electrodes can be mounted on the balloon surface, providing electroporation when the balloon is inflated and in contact with vessel walls.

In some examples, the systems and methods herein may include wire-guided catheters. These catheters are inserted over a guide wire. Incorporating electrodes along the catheter body or at the tip can allow for electroporation along the insertion path or at the target location.

In these or other examples, the catheter, wire, or other delivery system may be equipped with sensors (e.g., pressure, temperature) that may also house electrodes. This integration allows for real-time monitoring of tissue response to electroporation, enhancing treatment precision. In still other examples, multiple catheters, tubes, or wires, may be delivered coaxially, in a nested fashion (similar to an antenna). This may include an inner and outer tube, where the inner tube can be used for drug delivery or other purposes, while the outer tube houses electrodes for electroporation, or any other configuration. Each of these catheter designs can be tailored with the previously described electrode configurations to create a comprehensive system capable of addressing a wide range of medical applications, particularly in infection control and treatment.

Optionally, a variety of enhancements to catheter designs may be included in the systems and methods herein that are aimed at maximizing the efficacy and specificity of electroporation for infection prevention and treatment. In some examples, this may include catheters designed with flexible or adjustable geometries. Such designs could adapt to the varying anatomical structures within patients, ensuring optimal contact and electroporation effectiveness. This adaptability enhances the precision of bacterial cell targeting, reducing the risk of infection without compromising catheter functionality. In some examples, catheters with specialized surface textures, such as micro-grooves or nanopatterns, increase the surface area in contact with bacterial biofilms. These textures can facilitate more uniform and effective distribution of electric fields, thereby improving the disruption of biofilms and enhancing the catheter's antimicrobial properties.

Still other examples include the incorporation of smart materials into catheter designs. These materials can change properties (e.g., shape, stiffness) in response to electrical stimuli, allowing for dynamic adjustments of the catheter's positioning or its interaction with tissue during electroporation. Such capabilities enable precise delivery of electroporation therapy, tailored to the specific needs of the treatment site. In another example, the deployment of micro or nanoscale electrodes on the catheter surface offers targeted electroporation capabilities. By focusing the electroporation effect directly where needed, these electrodes can minimize unintended impacts on adjacent tissues. This precision ensures a concentrated antimicrobial effect against pathogens with minimal side effects, promoting patient safety and recovery.

Optionally, the systems and methods herein may include a sophisticated control mechanism that can adjust various parameters to optimize the electroporation process for different medical scenarios. This control unit may be used to control waveform selection. The control unit can select from various waveforms, including square wave, exponential decay, biphasic pulses, or others, each suitable for different types of cells and infection scenarios. Alternatively or in addition, the control unit may select intensity and duration. The control unit may allow for the adjustment of pulse intensity and duration to ensure effective electroporation while minimizing potential tissue damage. In this or other examples, the control unit may implement real-time feedback. Equipped with sensors, the control unit can provide real-time feedback on the electric field strength and tissue response, allowing for dynamic manual or automatic adjustments during the procedure.

Integrated System: For PICCs and peripheral IVs, electroporation electrodes can be embedded directly into the catheter walls. These catheters can then be connected to an external generator when electroporation is required.

In one example, the catheter or electroporation member may have a self-contained control unit. This example may include a self-contained power source and control unit, making them independent and more convenient for routine sterilization. In another example, an additional, specialized electroporation catheter can be used alongside standard medical catheters. This design allows for electroporation without modifying existing catheter designs. The tandem catheter can be positioned precisely where infection risk is highest, providing targeted electroporation treatment.

In these or other examples, treatment protocols can be pre-programmed based on the type of catheter, infection risk, and patient-specific factors. Alternatively, or in addition, the system can operate automatically based on these protocols, reducing the need for constant manual adjustments. The system can be controlled remotely, allowing healthcare providers to activate or adjust electroporation settings without direct physical interaction. Alternatively, the system may be activated and/or controlled directly at the bedside. In some examples, continuous recording of electroporation parameters and tissue responses for analysis and improvement of treatment protocols. These and other examples may include optimization of the electroporation parameters, such as voltage, pulse duration, frequency, and/or waveform. Further examples may include adaptive control mechanisms and safety thresholds. In these and other examples, the systems and methods herein may include algorithms for customizing electroporation parameters (e.g., intensity, frequency, pulse duration) based on real-time data from sensors integrated into the catheter system. This approach allows for adaptive treatments that can respond to changes in infection severity or tissue response, optimizing therapeutic outcomes.

Other examples of the systems and methods described herein may include mechanisms for abscess control or destroying tumors. This may include electrodes, bipolar, monopolar, or otherwise that are capable of ablating or destroying tissue according to the specifications of the control unit. In these and other examples, this may include a mechanism of clotting blood at the tip of or along the catheter or other member. The implementation of this electroporation system in medical catheters offers a versatile and effective approach to combat catheter-related infections, significantly enhancing patient safety and care quality.

In view of the foregoing, the present application also contemplates additional examples of device functionality, including broader integration features and enhanced safety measures that may further optimize infection prevention or treatment by electroporation. In some examples, the systems may incorporate real-time bacterial monitoring via sensors positioned along or within the catheter assembly. These sensors may detect biofilm formation, pH shifts, or impedance changes indicative of microbial presence, allowing the control unit to trigger electroporation pulses automatically or provide alerts to clinicians for timely intervention. In related examples, the sensors may also measure local temperature or tissue integrity during pulsed field delivery, enabling closed-loop control that tailors pulse duration, amplitude, and frequency based on immediate feedback.

To expand therapeutic flexibility, certain examples may include multiple medication compartments integrated into or adjacent the electroporation catheter or implantable device. Each compartment could separately store an antibiotic, anticoagulant, diuretic, antiviral, or other agent. The control system may selectively draw from one or more compartments in response to infection risk, hemodynamic changes, or sensor-detected parameters. In some configurations, electroporation pulses may temporarily increase cell permeability to locally enhance delivery of the stored drugs, further augmenting the antimicrobial or therapeutic effect.

In additional examples, the electroporation unit may operate in synergy with antibiotic lock solutions or antimicrobial coatings on the catheter surfaces. For instance, short, prophylactic electroporation cycles may be programmed at set intervals to disrupt early-stage bacterial colonies or biofilms, while conventional chemical treatments remain in place. Similarly, an optional tandem design may incorporate a secondary lumen or external wrap dedicated solely to electroporation electrodes, which can be activated or replaced independently of the primary infusion lumen.

The present application provides an electrode assembly for implantable devices wherein one or more electrodes are coated with a catalytic or enzyme-mimetic material. The coating is designed to enhance the generation of H₂O₂ when a controlled electrical potential is applied at the electrode-cathode in the presence of dissolved oxygen. In one example, the coating comprises a catalytic material selected from noble metals (e.g., platinum, gold) or transition metal oxides. In another example, the coating comprises an enzyme-mimetic compound (nanozyme) that emulates oxidase activity, thereby promoting the efficient reduction of oxygen to H₂O₂.

The system may be implemented on pacemaker leads, catheters, or other implantable devices. By controlling the electrical parameters (voltage, pulse duration, and waveform), the system achieves localized H₂O₂ generation at concentrations effective for biofilm disruption while remaining within safe limits for surrounding tissues. This approach minimizes the need for device extraction and offers a potential therapeutic strategy for chronic implant-related infections.

Still other examples may integrate advanced waveform control, such as biphasic or multi-phase pulses, for improved bacterial cell disruption and minimal collateral impact on surrounding tissue. The electroporation assembly may further include ring-based or matrix-based electrodes spaced at regular intervals to ensure uniform coverage along the catheter's length, or a segmented electrode design that targets specific areas of known higher infection risk (e.g., near the insertion site or at a bifurcation). These electrode arrangements may be powered by an on-board energy store (e.g., a rechargeable capacitor bank) or through an external power coupling that delivers high-voltage pulses when needed, reducing the size of any permanently implanted battery.

In some examples, the system is designed for seamless integration with existing implantable cardiac devices or patient-monitoring equipment. For instance, lead wires from a pacemaker or defibrillator may piggyback certain low-power signals to the electroporation module for synchronization or to initiate sterilization routines upon detecting arrhythmias or suspected sepsis. Alternatively, software algorithms running within the device's control unit could coordinate all electroporation cycles based on scheduled drug administration, sensor data, or external commands from a clinician's programming console. The console may log device performance data, bacterial load trends, or pulse efficacy results, enabling iterative fine-tuning of the treatment protocols over time.

Described herein are examples of intravenous (IV) catheter systems and methods for prolonging dwell time and reducing catheter-related complications through the use of pulsed field ablation (PFA), also known as electroporation-based therapy. The catheter systems may further incorporate auxiliary modalities such as ultraviolet (UV) light, specialized ionic solutions, and thermal modulation (heat or cold) to enhance antimicrobial efficacy, inhibit thrombosis, and mitigate inflammatory responses.

In one example, the system comprises an IV catheter having a flexible cannula formed of a biocompatible polymer, such as polyurethane or polytetrafluoroethylene. Embedded within or affixed to the cannula are one or more electrodes configured to deliver pulsed electrical fields to the surrounding tissue, fluid, or both. The electrodes are connected via conductors extending from the distal or mid-length regions of the catheter to an electrical interface located proximate the catheter's proximal hub. This interface couples to an external pulse generator, which provides the requisite electrical pulses for PFA.

During operation, the external pulse generator applies short, high-voltage or moderate-voltage pulses that cause temporary or irreversible electroporation of microbial cell membranes. By selectively disrupting bacterial or fungal cells adhering to the catheter surface or in the immediate vicinity, the system reduces biofilm formation and thereby lowers infection risk. PFA may be delivered at predetermined intervals (e.g., hourly, daily) or triggered based on sensor feedback, such as impedance measurements indicating early-stage microbial colonization or fibrin sheath formation.

PFA reduces the microbial load on the catheter, mitigating infection-driven catheter failure. In certain implementations, the catheter's electrodes or adjacent surfaces may be coated or impregnated with antimicrobial agents (e.g., silver ions or chlorhexidine) to further enhance bacterial disruption when used in concert with electroporation. The short, non-thermal nature of PFA pulses may minimize damage to endothelial cells, limiting local inflammatory responses. Intermittent or low-level pulses can be applied to modulate cell membrane permeability without inducing significant thermal injury, thereby reducing the likelihood of phlebitis. By preventing biofilm formation and maintaining healthier endothelium, PFA can indirectly decrease thrombus development. Additionally, specialized pulse protocols or flushes (described below) may further inhibit clotting.

In another example, the IV catheter includes a UV emission element, such as a UV-C light source (LED or fiber-optic based) arranged near or within the catheter hub. This light source delivers controlled doses of UV radiation to the catheter lumen and, to a limited extent, the pericatheter region. UV energy disrupts nucleic acids in microorganisms, providing a secondary antimicrobial effect complementary to PFA. UV bursts can be synchronized or alternated with PFA pulses. For example, a short cycle of UV exposure may occur immediately before or after an electroporation session, maximizing microbial disruption. The catheter assembly may include opaque or reflective coatings to confine UV light to the catheter lumen or targeted tissue zones, preventing unintended exposure of patient tissues or clinical personnel.

In certain configurations, the system employs specialized saline solutions or flush solutions containing adjusted ionic compositions. Examples include silver ions, zinc ions, or other biocompatible cations known to have antimicrobial or anti-inflammatory properties. The ionic concentrations may be selected to optimize electroporation efficacy by increasing or tuning the electrical conductivity in the vicinity of the electrodes. The catheter may be periodically flushed with the specialized solution prior to or during pulsed field delivery. This ensures that the local environment supports efficient and uniform electric field distribution. The specialized flush can be administered in a way that does not interfere with concurrent IV medications. Compatibility tests and in-line filtration systems may be used to safeguard against precipitation or chemical reactions.

Another optional feature includes temperature control near the catheter tip, which can be achieved by circulating fluid at selected temperatures through a dedicated lumen or by integrating thermoelectric elements in the catheter design. Mild hyperthermia may improve local blood flow, enhancing immune cell access and potentially synergizing with the electroporation mechanism. A small heating coil or thermoelectric module can be embedded in the catheter or housed in an external cartridge that circulates warmed flush fluid. Hypothermia may reduce inflammation and edema around the insertion site, potentially minimizing phlebitis. Coolant fluid or a thermoelectric cooling system can reduce the temperature locally, either before or after delivering a PFA cycle.

For further optimization, the system may incorporate impedance sensing electrodes or other micro-sensors (e.g., temperature or pressure). These sensors may monitor physiological changes in the tissue environment: Fluctuations in impedance may signify early biofilm formation, partial occlusion, or tissue inflammation. When readings exceed a predefined threshold, the controller may initiate a PFA cycle or adjust the pulse parameters to address the emerging complication. For devices using thermal modulation, an embedded thermistor or infrared sensor ensures that any heat or cold application stays within safe limits.

A healthcare professional inserts the IV catheter into the patient's vasculature using standard sterile technique. Once in place, the catheter is secured, and the electrode connectors are attached to an external pulse generator and, if present, the UV source or thermal control system. Prior to fluid administration, the controller may run a quick impedance sweep or test pulse to confirm electrode functionality and establish a baseline.

The catheter is then used to deliver fluids or medications. Periodically, a specialized flush containing ionic additives can be introduced to maintain optimal conditions for subsequent PFA treatments. At predefined intervals (e.g., every 12 hours) or upon detection of significant impedance changes, the system triggers pulsed field ablation. Electroporation pulses are applied over a short duration (microseconds to milliseconds per pulse), minimizing patient discomfort or disruption of infusion flow. UV light may be activated briefly after the PFA session to eradicate any remaining or weakened microorganisms in the lumen. If equipped, mild warming or cooling is applied for a set period to further discourage microbial growth or alleviate local inflammation.

All sensor data—impedance, temperature, UV on/off cycles, pulse parameters—may be recorded by a controller for ongoing analysis. Clinicians can review this information to determine whether the catheter remains viable or if an earlier replacement is advisable. By reducing infection risk, controlling inflammation, and mitigating thrombotic complications, the overall dwell time for the IV catheter is prolonged. This can decrease the need for frequent catheter replacements and reduce patient discomfort.

PFA in combination with UV light and ionic solutions provides robust defense against bacterial or fungal colonization. The non-thermal nature of PFA coupled with potential thermal management (if implemented) helps limit endothelial damage and subsequent inflammation. Automated or semi-automated operation allows clinicians to focus on other patient needs without extensive manual interventions. Fewer catheter replacements and fewer complications can lead to improved patient comfort and reduced healthcare costs.

The described IV catheter system integrates pulsed field ablation with optional UV disinfection, specialized ionic flush solutions, and thermal modulation to extend catheter life and enhance patient safety. By combining multiple synergistic methods of microbial control and vascular health maintenance, the catheter system can significantly reduce the incidence of infection, phlebitis, and other mechanical or physiological failures typically associated with indwelling intravenous access.

Through these and other enhancements, the systems and methods disclosed herein may offer a comprehensive solution for both preventing and managing infections associated with indwelling medical catheters or implantable devices. By integrating continuous or on-demand electroporation capabilities, targeted drug delivery, and adaptive sensor feedback, the systems aim to minimize catheter-related complications and improve long-term patient outcomes.

### BRIEF DESCRIPTION OF THE DRAWINGS

The invention is best understood from the following detailed description when read in conjunction with the accompanying drawings. It will be appreciated that the exemplary apparatus shown in the drawings are not necessarily drawn to scale, with emphasis instead being placed on illustrating the various examples and features of the illustrated examples.

FIG. 1 shows an example of a system implanted within a patient's body into the bladder and exhibiting a Foley catheter or similar.

FIG. 2 shows another example of a system implanted within a patient's body, in the vasculature, leading to the heart, exhibiting a PICC or similar.

FIG. 3 illustrates a dialysis catheter system incorporating electroporation electrodes positioned along the catheter near the insertion site to prevent bacterial infections, with a power source supplying controlled electrical pulses to disrupt microbial biofilms while maintaining vascular access for hemodialysis.

FIGS. 4A and 4B illustrate a port system (port-a-cath) with an electroporation-enabled catheter positioned within the vasculature for drug delivery.

FIG. 5 illustrates an implantable pacemaker or ICD system with electroporation functionality, showing a generator, lead placement, and fixation mechanisms for stable pacing and infection control.

FIG. 6 provides a close-up view of the pacemaker lead system, highlighting the coiled lead storage within the generator, an expandable fixation member, and embedded electroporation electrodes along the lead.

FIG. 7 depicts an alternative pacemaker design where the lead coils like a telephone wire, integrating an external power access site for electroporation activation.

FIG. 8 presents an ICD or pacemaker system where the lead coils around the generator externally, featuring tripod-like stabilization legs that secure the lead within the heart.

FIG. 9 provides a detailed view of the generator, lead structure, and tripod-like stabilization mechanism, highlighting pacing electrodes and electroporation-enabled components for infection prevention.

FIG. 10 illustrates external energy delivery to an electroporation-enabled pacemaker or ICD system, showing a percutaneous power probe, monitoring system, and electrical field generation around the implanted generator.

FIG. 11 depicts a large drug reservoir system with a flexible storage pouch, a delivery catheter, and an implantable generator that may supply power for electroporation-assisted drug release.

FIGS. 12A-12F illustrate various electroporation pulse waveforms, including biphasic, stepped, sinusoidal, and exponential decay patterns, which can be used to control membrane permeability and optimize bacterial disruption.

FIGS. 13A-13F show asymmetric and monophasic variations of electroporation waveforms, demonstrating how polarity bias and unbalanced charge distributions can influence electroporation efficiency and directional effects.

FIGS. 14A-14E show a simplified representation of bacterial electroporation, showing how increasing electric field strength leads to progressive membrane disruption, pore formation, and eventual cell lysis.

### DETAILED DESCRIPTION

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

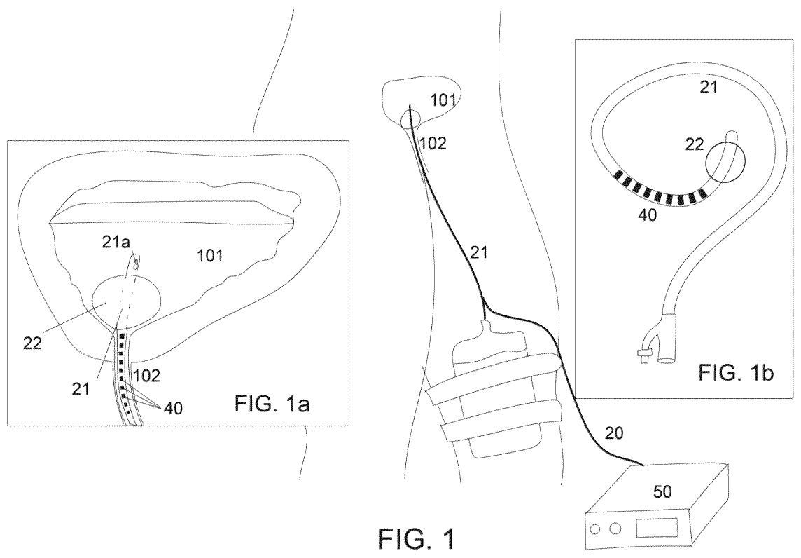

Turning to the drawings, FIG. 1 shows an example of a system 20 that may be implanted within a patient's body for the prevention and/or treatment of infection. In the example shown, a Foley catheter 21 is used for urinary drainage and inserted into the urethra 102 into the bladder 101 to drain urine. The catheter 21 includes a tubular member including a proximal end and a distal end carrying a plurality of electrodes and/or sensors 40, a balloon or other expanding mechanism 22, and including one or more lumen 21a. The electrodes and/or sensors 40 are connected to a control unit 50 for operating the system 20, e.g. to perform the various methods and/or functions described elsewhere herein.

Electroporation involves applying short bursts of electric fields to create transient pores in cell membranes. This process is typically reversible and does not cause lasting damage to healthy cells but can be lethal to bacterial cells. As shown, the proximal end of the catheter 21 couples to the control unit 50 to control the electrical pulses or fields to damage and/or kill bacterial cells without harming healthy cells. Electrodes 40 may be embedded in, on, or within the catheter 21, inserted through a lumen 21a, may be placed intermittently along the length of the member 21, or strategically placed at select locations.

Control unit 50 may implement one or more of different waveforms for electroporation. The system may employ various waveforms for effective electroporation, including: Square Wave Pulses, characterized by a constant amplitude for a specific duration, beneficial for creating uniform and controlled pores in cell membranes; Exponential Decay Pulses, which start at a high amplitude and decrease exponentially, useful for targeted electroporation with varying intensity; Biphasic Pulses, consisting of two phases, one positive and one negative, which help in reducing electrode erosion and can be tuned for specific cell types, among others. The system's control unit can select and vary these waveforms based on the application requirement, ensuring optimal electroporation for infection control. Alternatively, or in addition, the electrodes 40 may include sensing mechanisms, such as ultrasonic, pressure, optical, magnetic, impedance, resistance, piezoelectric, or other types of sensing. These sensors may communicate with controller 50 to implement automatic detection of bacteria and implement necessary electrical fields or pulses.

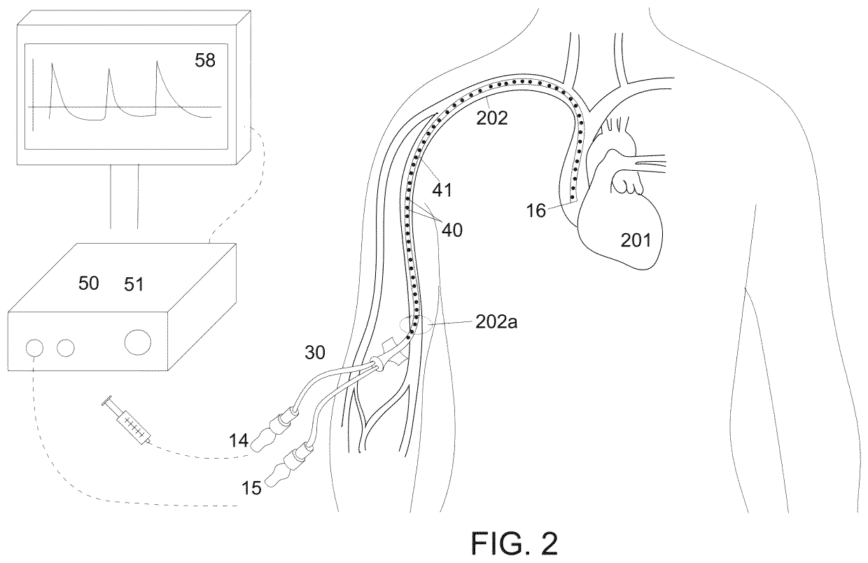

In FIG. 2, an example is shown wherein the system 30 is inserted into a vessel 202 at insertion site 202a leading to the heart 201. In this example, member 41 is a Peripheral Intravenous Catheters (PICCs) or peripheral IV, which can be a conduit for pathogens to enter the bloodstream, leading to infections like bacteremia or sepsis. The catheter 41 may include electrodes and/or sensors 40, lining the member or specifically concentrated at insertion site 202a to prevent or treat bacterial infection. The catheter connects to the control unit (not shown) for automatic or manual control as described elsewhere herein. Alternatively or in addition, the control and power of the electroporation mechanism may be self-contained on or within the catheter itself.

In these or any other examples, the catheter 41 or the member with the embedded electrodes/and or sensors may be configured to be steerable, flexible, or shape-changing to optimally reach desired locations. This may include automatic deployment and adjustment using sensing, or manual adjustment through operator actuation. The system 30 includes a flexible cannula 41 made from a biocompatible polymer (e.g., polyurethane, PTFE). A hub 14 is connected at the proximal end of the cannula 41, allowing standard IV fluid administration. The distal tip 16 may be tapered or beveled for vascular insertion.

Embedded within or applied along the cannula wall are one or more electrodes 40. These electrodes deliver pulsed electrical energy (e.g., for electroporation) and can also function as sensing elements for impedance measurements. In some examples, at least one electrode is positioned near the distal tip to optimize both treatment delivery and localized impedance assessment.

Optionally, the catheter may further include: A UV light source (e.g., a UV LED) integrated into or near the hub to project UV radiation into the catheter lumen or the pericatheter space; A specialized ionic coating or lumen flush solution formulated to optimize electrical conductivity for PFA and provide additional antimicrobial activity (e.g., silver or zinc ions); and A thermal control mechanism (e.g., a thermoelectric module or circulating fluid pathway) for mild heating or cooling at or near the catheter tip.

These additional features aim to synergize with the pulsed electrical fields, reducing microbial colonization, mitigating inflammatory response, and thereby extending the catheter's functional duration.

An electrical interface 15 connects the electrodes 40 to an external power source 50 and an impedance-measuring module 51 (which may be integrated with or separate from the power source). When the catheter is in situ, the electrodes 40 can deliver pulsed field ablation energy according to user-defined parameters or automated protocols. Additionally, these same electrodes periodically (or continuously) measure electrical impedance in the tissue and fluid environment surrounding the catheter.

Impedance-Measuring Module 51: This module applies a low-level excitation signal and measures voltage and/or current changes to determine the impedance near the catheter tip or along the cannula. The measured impedance value is relayed to a controller 58, which interprets shifts in impedance as potential signs of bacterial biofilm formation, fibrin sheath development, or significant changes in the tissue environment (e.g., infiltration or inflammation).

When specialized ionic solutions are utilized, the impedance-measuring module may also detect variations in conductivity related to the infused solution's ion concentration. This real-time feedback can guide the dosing or timing of PFA and help optimize the electric field distribution for microbial eradication.

The power source 50 includes a pulse generator capable of delivering pulsed electrical energy in the form of square waves, sine waves, bipolar pulses, or other waveforms. Pulse parameters such as voltage, current, frequency, pulse duration, and duty cycle can be pre-set or dynamically adjusted. In one example, the controller 58 automatically initiates a PFA sequence when the impedance measurement indicates early-stage biofilm growth or occlusion. Medical staff may also trigger PFA sessions on demand, for instance, if local infection or catheter malfunction is suspected.

In examples featuring integrated UV light sources, the controller 58 may synchronize the pulsed field delivery with short UV radiation bursts to achieve a combined antimicrobial effect. If the system includes thermal modulation, the controller can regulate mild heating or cooling cycles before, during, or after PFA pulses to further curb inflammatory responses or enhance membrane permeability.

The catheter is inserted using standard venipuncture, and the electrodes are connected to the external power source 50 and impedance module 51 via the hub's electrical interface. The catheter is used for routine IV infusions and drug delivery. At scheduled intervals (e.g., hourly, daily) or continuously, low-level electrical signals are passed through the electrodes to measure local impedance. When the impedance data suggests a threshold has been exceeded—implying microbial colonization, clot formation, or significant tissue reaction—the system (or an operator) can initiate pulsed field ablation.

Where applicable, the system may also: activate a UV emitter for a brief period to disinfect the catheter lumen or nearby fluid; introduce a specialized ionic flush into the lumen to optimize conductivity and antimicrobial properties; and/or enable mild heating or cooling at the catheter tip to modulate inflammation and blood flow.

Impedance trends can be recorded over time. A sustained rise or erratic fluctuation in impedance may signal that the catheter has reached the end of its functional life, guiding proactive replacement before complications arise.

Localized delivery of pulsed fields directly at the site of microbial colonization. Impedance measurements provide an objective metric to assess catheter patency, infection risk, and tissue health. Regularly timed or on-demand PFA treatments, triggered by impedance changes, reduce infection risk and inflammation, prolonging catheter usability. Combines sensing and treatment in a single device, reducing complexity and procedural burden. The optional addition of UV disinfection, ionic solutions, and thermal modulation enhances antimicrobial efficacy, mitigates local vein irritation, and furthers the goal of preserving catheter function.

In another example, the catheter or a member such as a wire, needle, loop, tube, or otherwise may be lined with electrodes and/or sensors that travel into the heart to treat endocarditis. The distal end of the member may include a balloon, or may take on a shape such as a loop, sphere, or other shape to increase surface area for application of electric fields to infection. The electric pulses may be controlled automatically based on time or sensor data or manually by the physician.

In some examples, the electroporation electrodes and sensors 40 are combined with a catheter 41 embedded within, in, or on the member itself. In other examples, the electrodes/sensors 40 are included on a separate member (not shown) that may be deployed through lumens 41a or beside catheter 41 in tandem with the catheter. The pulses delivered may be capable of delivering high voltage for short durations to target bacterial cells, or may be adjusted for duration, voltage, and/or waveform to target other specific types or tissue or cells found in abscesses, tumors, or otherwise.

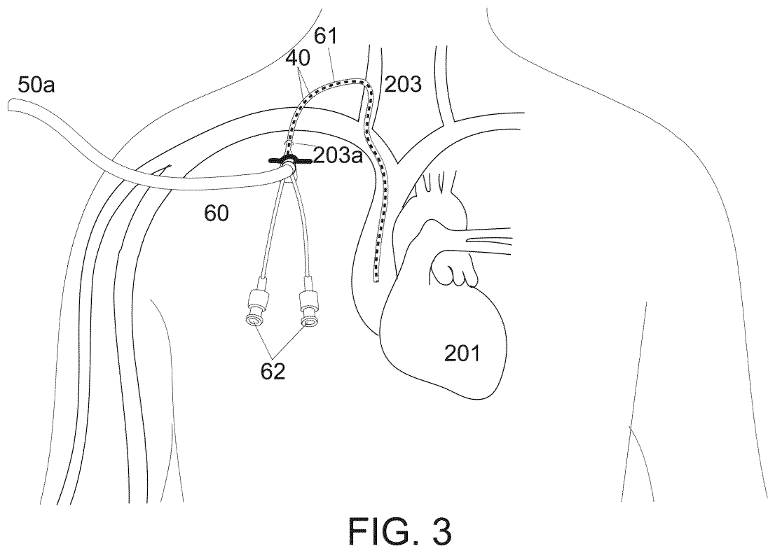

FIG. 3 illustrates an example of a dialysis catheter system 60 with elongate member 61 configured for infection prevention and/or treatment using electroporation. The catheter 61 is inserted into a vessel 203 at an insertion site 203a leading towards the heart 201, ensuring vascular access for dialysis treatment while minimizing the risk of bloodstream infections, such as bacteremia or sepsis.

The system includes a series of electrodes 40 positioned along the catheter, particularly near the insertion site 203a where infection risk is highest due to prolonged vascular access. These electrodes are capable of delivering pulsed electric fields to disrupt bacterial cell membranes, preventing microbial colonization without harming healthy tissues.

The catheter system 60 features dialysis ports 62, which serve as access points for blood withdrawal and return during hemodialysis. These ports are essential for efficient blood filtration while maintaining sterility. The system is connected via a power connector 50a to an external power source 50 (not pictured), which supplies energy for electroporation pulses. The power source may include a pulse generator capable of delivering controlled electrical fields, with parameters such as voltage, frequency, pulse duration, and waveform adjusted manually or automatically.

Each electrode 40 may be wired individually or in various configurations to optimize electroporation efficacy. Individually Addressable Electrodes: Each electrode 40 is independently wired and controlled, allowing for localized electroporation treatment at specific sites. The control unit 58 (not pictured) can selectively activate individual electrodes based on real-time impedance measurements or sensor feedback, ensuring precise bacterial eradication. Paired or Grouped Electrodes: Electrodes may be configured in pairs or sets along the catheter to generate a uniform electric field around the insertion site 203a. This method reduces power consumption while still providing effective microbial disruption. Alternating Electrode Activation (Multiplexing): The system may alternate electrode activation in a sequential or rotating pattern, preventing localized heating while ensuring comprehensive field coverage. This approach is particularly useful for prolonged catheter use, as it maintains electroporation effectiveness without causing electrode degradation. Bipolar vs. Monopolar Configurations: In a bipolar setup, two adjacent electrodes function as anode and cathode, creating a localized electric field between them. In a monopolar setup, one electrode on the catheter serves as the active element, while a remote ground pad (external or implanted) completes the circuit. Bipolar configurations are well-suited for localized electroporation near the catheter, whereas monopolar configurations can extend the field over a larger area. Capacitive Coupling for Non-Contact Electroporation: Electrodes may use capacitive coupling, where electric fields are applied without direct electrode contact with blood or tissue. This non-invasive approach minimizes the risk of electrode fouling while still disrupting bacterial biofilms.

Impedance sensors integrated within or near the electrodes can detect early biofilm formation or changes in blood conductivity, triggering electroporation cycles automatically. The control unit 58 and power generator 50 may also adjust electroporation intensity in response to sensor feedback, ensuring safe and effective treatment while minimizing tissue irritation.

This dialysis catheter system 60 integrates electroporation-based bacterial control with conventional dialysis functionality. The strategic placement and flexible wiring configurations of electrodes 40 ensure precise, controlled antimicrobial treatment, reducing the likelihood of bloodstream infections. The power connector 50a links to an external power source 50, which delivers optimized pulses through the individual or grouped electrodes based on real-time feedback.

In one example, electroporation technology is integrated into dialysis ports to actively prevent and treat bacterial infections, reducing the risk of catheter-related bloodstream infections (CRBSIs). The system incorporates electrodes and sensors at multiple locations, including just beneath the skin, along the catheter's length, and at the catheter tip, ensuring comprehensive coverage of potential infection sites. A set of conductive electrodes may be embedded subcutaneously at the port site, arranged in a concentric ring pattern around the access point. These electrodes generate electric fields that radiate outward, targeting bacteria that have colonized the tissue surrounding the insertion site. To ensure effective application, the electrodes may be printed onto a flexible, biocompatible polymer substrate that conforms to the contours of the patient's skin and underlying structures. Integrated impedance sensors may detect fluctuations in tissue conductivity, signaling early bacterial colonization and automatically triggering electroporation treatment.

Along the catheter's length, a series of ring electrodes may be embedded into the catheter wall at intervals, allowing for localized electroporation activation at high-risk sites. The electrodes may be selectively engaged based on sensor feedback, ensuring treatment is applied only where bacterial biofilm accumulation is detected. To enhance efficacy, the catheter may include a conductive nano-coating that promotes uniform electric field distribution when pulses are applied. At the catheter tip, an electrode positioned at the distal end may periodically emit electroporation pulses to prevent bacterial migration into the bloodstream. In some configurations, the catheter tip may incorporate an expandable balloon or a deployable mesh structure that increases the contact area for electric field application, further enhancing bacterial membrane disruption.

To provide additional protection at the skin entry site, an external electroporation-enabled cover may be positioned over the catheter insertion point. In one variation, a flexible, disposable patch containing embedded microelectrodes may adhere to the skin surrounding the dialysis port. This patch may generate electroporation fields that penetrate the superficial tissue layers, eradicating bacteria at and just beneath the skin surface. The patch may also incorporate real-time bacterial load sensors that trigger treatment when microbial presence exceeds a predetermined threshold. In another example, a biocompatible hydrogel containing conductive nanoparticles or embedded microelectrodes may be applied directly to the insertion site, serving both as a physical barrier to bacterial ingress and as a medium for efficient electric field transmission. This hydrogel may include embedded sensors that detect microbial activity and dynamically adjust electroporation parameters to optimize bacterial eradication.

Alternatively, a reusable, snap-on cap with integrated electrodes may be placed over the dialysis port when not in use. This cap may wirelessly receive power to deliver periodic electroporation pulses, preventing bacterial ingress between dialysis sessions. In some implementations, the cap may contain antimicrobial agents that synergistically enhance the effects of electroporation-induced bacterial membrane disruption. The electroporation system may be powered through an implantable rechargeable battery, which can be replenished using wireless energy transfer, or through an external wearable controller that patients can use to activate sterilization cycles as needed.

In some examples, electroporation may be used in conjunction with antimicrobial treatments to enhance infection control. Low-intensity electroporation pulses may be applied while an antibiotic lock solution is infused into the catheter, temporarily increasing bacterial permeability and improving drug penetration into biofilms. Additionally, the system may incorporate photoclectroporation, combining electroporation with light-based sterilization, where an external patch or catheter-integrated sensors trigger antimicrobial UV or blue-light exposure in response to bacterial detection. By integrating electroporation at multiple points—including beneath the skin, along the catheter, and at the insertion site—this system provides a multi-layered defense against bacterial infections in dialysis ports. The combination of subcutaneous electrodes, catheter-embedded electroporation arrays, external antimicrobial patches, and real-time sensor feedback offers an adaptive and proactive infection control strategy, improving patient outcomes and extending the longevity of dialysis access devices.

In some examples, an electroporation or pulsed field ablation (PFA) system may be incorporated into an elongate member positioned within a biologic conduit configured to exchange fluid. Such a system may be deployed in dialysis catheters, arteriovenous (AV) grafts, hemodialysis access sites, peritoneal dialysis catheters, or vascular conduits to facilitate sterility, biofilm disruption, and solute exchange enhancement. The system may include permanent, semi-permanent, or removable electrodes, integrated within or around the fluid-exchange member, to selectively apply pulsed electric fields for bacterial eradication, biofilm prevention, or modulation of fluid transport properties.

In one example, the elongate member may comprise a dual-lumen dialysis catheter, wherein electroporation electrodes are integrated into one or both lumens to prevent bacterial colonization along blood-contact surfaces. The electrodes may be positioned in intermittent ring patterns, as linear strips along the inner catheter wall, or embedded within the catheter material itself. The electroporation pulses may be applied before, during, or after dialysis sessions to sterilize the catheter while minimizing disruption to blood flow dynamics.

In another example, the dialysis catheter may feature an external cuff with integrated electrodes, positioned at or near the exit site of the catheter to mitigate catheter-related bloodstream infections (CRBSIs). This cuff may deliver periodic or on-demand pulsed field therapy, disrupting bacterial adhesion before biofilms can form. In some configurations, the electroporation electrodes may be selectively activated based on real-time bacterial detection, optimizing infection control while minimizing energy consumption.

In yet another variation, a deployable electroporation assembly may be introduced via a sheath or guidewire to temporarily conform around a hemodialysis catheter, delivering short-duration electric pulses before being retracted or removed. This approach allows periodic biofilm disruption without requiring permanent modifications to existing dialysis catheters, making it compatible with standard clinical protocols.

In some examples, the electroporation system may be applied to AV grafts, fistulas, or synthetic vascular shunts, wherein electrodes are embedded within or around the graft wall. This system may deliver localized electric pulses to: inhibit bacterial colonization on graft surfaces; reduce neointimal hyperplasia, which can contribute to graft stenosis; and/or prevent thrombosis by modulating endothelial function. In one example, a flexible electrode mesh may be deployed through the graft lumen, temporarily conforming to the inner surface to apply pulsed field therapy for biofilm eradication and tissue modulation. This system may be designed for periodic treatment sessions, allowing for sterile maintenance without surgical intervention.

In another example, an electroporation-based system may be incorporated into peritoneal dialysis catheters to enhance sterility and solute transport across the peritoneal membrane. The electroporation electrodes may be positioned: along the catheter's outer surface, applying pulses to disrupt bacterial biofilms; internally within the catheter lumen, sterilizing fluid pathways during or between dialysis cycles; and/or at the catheter tip or fenestrations, where bacterial contamination is most likely to occur.

In some configurations, electroporation pulses may be used to transiently enhance membrane permeability, improving the diffusion of dialysate, waste products, or therapeutic agents across the peritoneal membrane. This effect may be used to accelerate dialysis efficiency, enhance ultrafiltration, or modulate localized tissue interactions. In another example, an expandable electrode sheath may be positioned around the peritoneal catheter, deploying radially arranged electrodes to sterilize the catheter's external surfaces while leaving it in place. This may reduce peritonitis risk without requiring catheter replacement or chemical disinfection.

In some examples, the electroporation system may be integrated with fluid exchange monitoring, allowing real-time modulation of electroporation parameters based on detected microbial load, biofilm formation, or solute clearance efficiency. The system may: adjust pulse intensity and duration in response to bacterial load detection; synchronize electroporation pulses with dialysis flow cycles, optimizing sterility without affecting fluid dynamics; and/or combine electroporation with chemical sterilization solutions, reducing the need for high-dose antibiotic lock therapy.

The electroporation system may also interface with dialysis machines, vascular graft sensors, or wearable health monitoring devices, allowing for automated control, remote activation, or integration into hospital-based dialysis protocols.

These configurations allow for electroporation-enhanced dialysis and fluid exchange applications, integrating sterility maintenance, biofilm prevention, and solute transport optimization across multiple vascular and peritoneal dialysis systems. By incorporating internal, external, and deployable electrode structures, the system provides versatile, programmable solutions for improving long-term device functionality while reducing infection risks in chronic kidney disease, hemodialysis, and peritoneal dialysis patients.

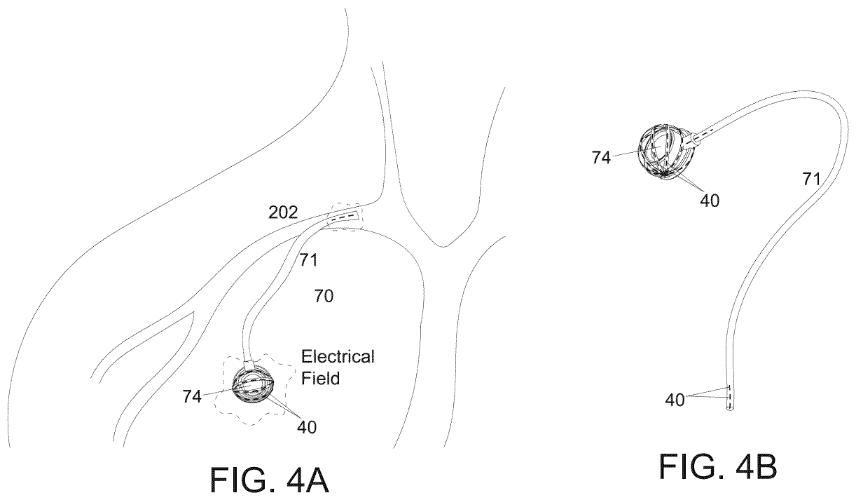

FIGS. 4A and 4B show an exemplary electroporation-integrated drug delivery port or port-a-cath system designed for infection prevention and vascular access. The system includes an implanted port 74 that is connected to a catheter 71, which extends into a blood vessel 202 to facilitate the delivery of therapeutic agents while minimizing the risk of bloodstream infections. Integrated electrodes 40 positioned along the catheter enable electroporation, generating an electrical field to disrupt bacterial membranes and prevent microbial colonization.

In FIG. 4A, the system is shown within the anatomical structure, where the catheter 71 is inserted into the vascular structure 202. The electrodes 40 are arranged in proximity to the catheter's insertion site to generate an electrical field that neutralizes bacteria before they can establish an infection. The system may include two distinct access points: one needle site for drug delivery and storage, allowing clinicians to administer chemotherapy, antibiotics, or other medications as needed, and another site dedicated to connecting power to the electroporation electrodes and sensors. This dual-access design ensures that electroporation can be activated independently from drug administration, allowing for targeted bacterial control while maintaining continuous or intermittent therapy.

FIG. 4B provides a more detailed perspective of the port 74 and catheter assembly, showing the configuration of the electroporation components. The electrodes 40 embedded along the catheter may be powered through an external connection to deliver controlled pulses, which can be adjusted based on real-time sensor feedback. The system may incorporate impedance sensors or biofilm-detection mechanisms to trigger electroporation cycles automatically, responding dynamically to bacterial presence.

Additionally, the electroporation-enabled chemo-port system may feature a conductive nano-coating on the catheter's inner and outer surfaces to enhance uniform electrical field distribution, ensuring effective microbial disruption without excessive heating or tissue damage. The system may also support monopolar or bipolar electrode configurations, where a monopolar setup utilizes an external grounding pad, and a bipolar setup generates a local electric field between adjacent electrodes.

By integrating electroporation at multiple points along the catheter and within the port itself, this system provides a multi-layered approach to infection control. The ability to independently control drug infusion and electroporation activation enhances flexibility in treatment protocols, allowing healthcare providers to manage infections proactively without interrupting drug delivery. The system's modular design supports various clinical applications, including chemotherapy, long-term antibiotic therapy, and total parenteral nutrition, while maintaining a sterile vascular access route.

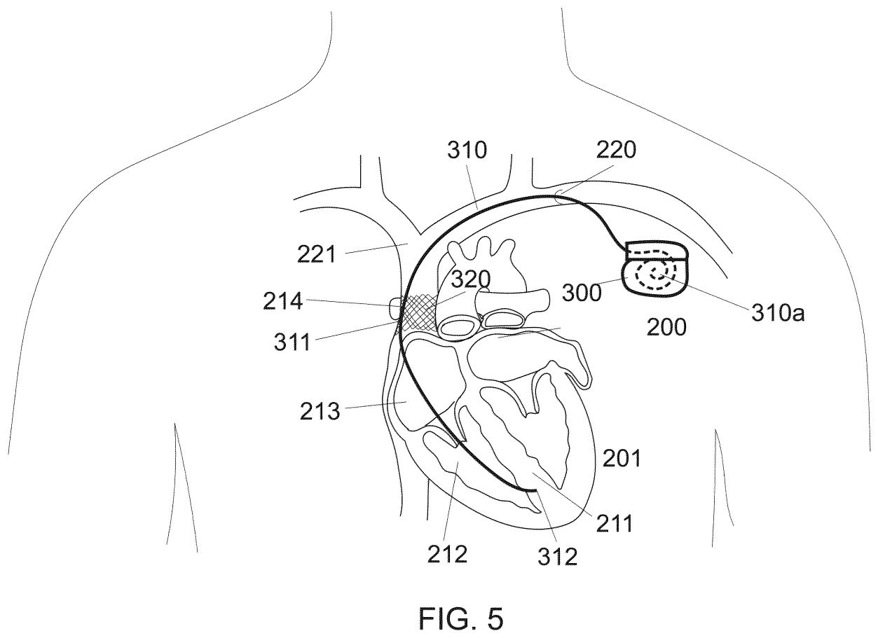

FIG. 5 illustrates a pacemaker system that integrates electroporation functionality to prevent infections and maintain lead sterility. The system includes a pacemaker generator (CAN) 300 implanted in a subcutaneous or submuscular location 200, with one or more leads 310 extending into the heart 201. These leads incorporate electrodes 311 and 312, which deliver cardiac pacing signals.

The pacemaker generator 300 is positioned in the pectoral region 200, with the lead entering the vessel 221 at the access site 220. The generator houses pulse-generation circuitry for cardiac rhythm management and may also contain electroporation circuitry capable of delivering pulsed electric fields to eliminate bacteria on or around the lead surfaces. The generator is enclosed in a biocompatible casing and powered by an internal battery. To manage excess slack, in some cases, the lead 310 may coil within the CAN 300 after insertion (310a coiled within CAN), preventing unwanted lead movement within the pectoral pocket 200.

The leads 310 extend from the generator into the vascular system 221 and terminate in the heart 201. Each lead consists of a conductor enclosed in a biocompatible insulating sheath, ensuring electrical safety and long-term durability. To facilitate electroporation, electrodes 40 (not shown) may be positioned along the leads to allow localized electric field application. These electrodes can be configured in different designs, including thin printed conductive layers of platinum or iridium along the sheath or discrete ring electrodes spaced at intervals along the lead. These configurations ensure uniform electroporation field coverage while maintaining lead flexibility.

An expandable member 320, made of braided nitinol or other expandable material, expands in the superior vena cava (SVC) to secure lead placement. This structure also ensures stable positioning of electrode 311 at the sinoatrial (SA) node 214 for pacing. The remainder of the lead passes through the right atrium 213 into the left ventricle 212, where electrode 312 is embedded in the septal wall 211 for additional pacing functions.

Electroporation energy may be delivered through two primary mechanisms. In one approach, a direct energy connection is established via a percutaneous access port, allowing healthcare providers to insert a sterile needle or probe to transmit high-voltage pulses to the electrodes 320. Alternatively, the pacemaker generator 300 may incorporate an integrated PFA circuit that automatically initiates electroporation upon infection detection. In another approach, wireless power transfer may be employed using an inductive coil within the generator, which receives energy from an external coil placed near the skin. This non-invasive charging method allows periodic recharging of the electroporation system without requiring direct physical connection.

To ensure timely activation of PFA, in some examples, the system integrates infection-detection sensors. Impedance sensors monitor changes in electrical resistance, which may indicate bacterial biofilm formation. Temperature sensors detect localized heating, signaling an inflammatory response or infection near the pacemaker leads. Based on this real-time data, the system can either automatically trigger electroporation treatment or allow manual activation via an external programmer.

In addition to electroporation, the system may employ synergistic antimicrobial strategies. The lead surface may be coated with antibiotic-impregnated polymers to inhibit bacterial adhesion. Furthermore, electroporation pulses can temporarily increase bacterial membrane permeability, enhancing the effectiveness of locally or systemically administered antibiotics. This combined approach reduces the risk of biofilm formation and minimizes antibiotic resistance development.

By integrating electroporation into pacemaker systems, this technology provides a proactive solution for preventing device-related infections, which are a leading cause of pacemaker explantation. Unlike thermal ablation methods, electroporation delivers precise bacterial cell disruption without excessive heat, minimizing tissue damage. Additionally, the inclusion of inductive recharging for PFA reduces the need for invasive procedures to maintain the system's functionality.

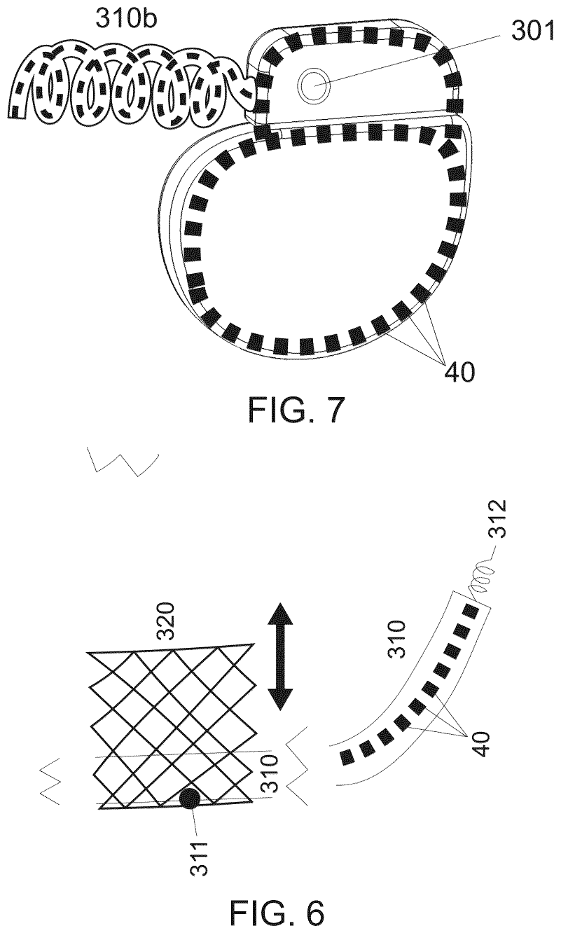

FIG. 6 provides a detailed view of key structural and functional components of the electroporation-enabled pacemaker system shown in FIG. 5. The illustration highlights the coiled lead storage within the pacemaker can 300, the expandable fixation member 320, and the electroporation electrode placements along the lead 310.

The top portion of FIG. 6 focuses on the pacemaker generator 300 and the coiling of excess lead 310a within the generator housing. This coiling prevents unnecessary lead slack in the pectoral pocket while maintaining flexibility for lead positioning. The lead 310 includes multiple electrodes 40, arranged along its length, which are capable of delivering both pacing pulses and electroporation energy to disrupt microbial biofilms.

The middle portion of FIG. 6 illustrates the expandable fixation member 320, designed to secure the lead 310 in the superior vena cava (SVC). This component, made of braided nitinol or other expandable materials, provides mechanical stabilization while housing electrode 311, which is positioned at the sinoatrial (SA) node. The expandable member ensures proper contact between the lead and the vessel wall, allowing for efficient pacing and electroporation delivery. The bidirectional arrow indicates the potential for controlled expansion or contraction, enabling customized lead positioning based on anatomical requirements.

The lower portion of FIG. 6 provides a magnified view of the lead 310 as it progresses toward the ventricular septum. Electrode 312 is embedded within the septal wall, ensuring stable pacing functionality. Additional electrodes 40 are spaced along the lead to facilitate localized electroporation. The arrangement of these electrodes allows for targeted electric field application, ensuring effective microbial disruption while minimizing unintended effects on surrounding cardiac tissues.

This close-up view demonstrates how the electroporation-enabled pacemaker system integrates both infection prevention and cardiac pacing functionalities. By incorporating strategically placed electrodes, coiled lead storage, and expandable fixation structures, the system enhances lead stability, reduces infection risk, and ensures long-term functionality within the cardiovascular system.

FIG. 7 illustrates an alternative configuration of the electroporation-enabled pacemaker system, in which the lead 310b adopts a coiled, telephone wire-like structure to enhance flexibility and strain relief. This design accommodates patient movement while maintaining optimal electrode positioning for both cardiac pacing and electroporation treatment.

The pacemaker generator 300 features an access site 301, which serves as a connection point for external power delivery. This port allows healthcare providers to supply electroporation energy when needed, either through a percutaneous connection or a wireless inductive charging interface. The ability to directly interface with the electroporation circuit enables controlled application of pulsed electric fields, ensuring targeted microbial disruption along the lead.

The coiled lead 310b improves adaptability by allowing extension and compression as the patient moves, reducing mechanical stress on the lead-body interface. Additionally, multiple electrodes 40 are embedded along the inner and outer surfaces of the pacemaker housing. These electrodes facilitate broad electroporation coverage, providing antimicrobial protection around the device pocket to prevent bacterial colonization.

This alternative design enhances durability, maintains secure lead positioning, and enables flexible electroporation energy delivery. By incorporating an external power access site, the system allows for both routine and on-demand electroporation treatments, improving long-term infection control in implantable pacemaker systems.

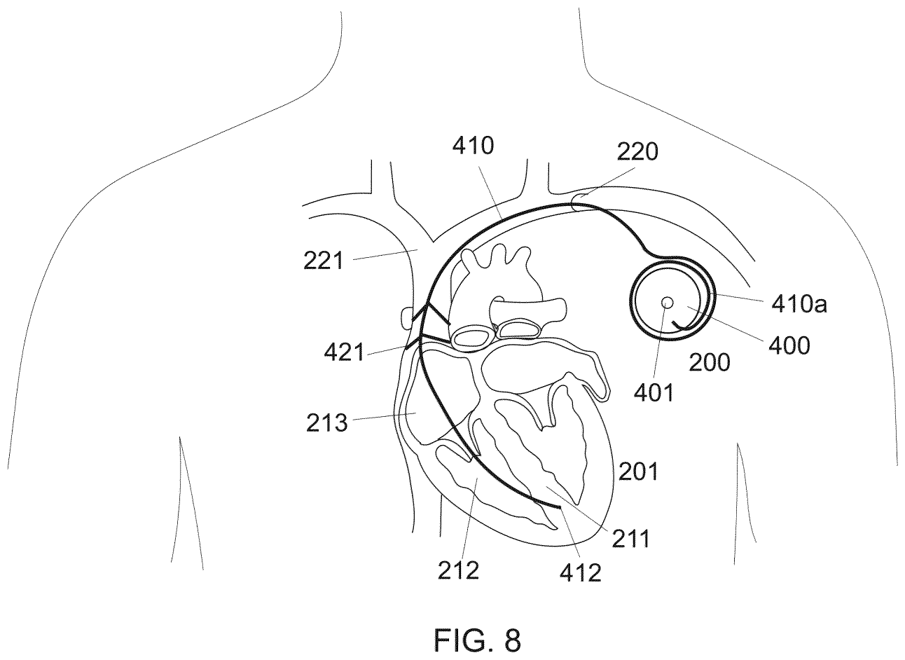

FIG. 8 illustrates an implantable cardioverter defibrillator (ICD) or pacemaker system with an alternative lead management and stabilization design. The system includes a generator 400, implanted in a subcutaneous or submuscular location 200, which houses pulse-generation circuitry for cardiac pacing and/or defibrillation, along with optional electroporation functionality for infection prevention.

In this configuration, the lead 410 extends from the generator 400 and enters the vascular system at access site 220, continuing through the superior vena cava (SVC) into the heart 201. Instead of coiling within the generator housing, the lead 410a now wraps around the external surface of the generator 400. This external coiling reduces excess slack in the pectoral pocket while maintaining flexibility and strain relief, preventing kinking or unnecessary movement that could disrupt lead positioning.

A key feature of this system is the inclusion of tripod-like stabilization legs 421 at a critical anchoring point within the heart. These legs secure the lead 410 in place, preventing migration due to cardiac motion or blood flow. The tripod-like structure ensures optimal electrode placement, improving both pacing and defibrillation effectiveness while maintaining stable electroporation field application.

Within the heart, the lead 410 extends through the right atrium 213 and terminates in the left ventricle 212, where electrode 412 is embedded in the septal wall 211 for pacing. The stabilization legs 421 keep the lead firmly in place at the point of contact, reducing mechanical dislodgement and ensuring long-term reliability.

An access port 401 is integrated into the generator 400, allowing for external power connection. This port can be used to deliver electroporation pulses for microbial disruption or to recharge the internal battery using inductive coupling. Additionally, antimicrobial coatings or embedded electrodes along the lead 410 may enhance infection prevention by applying pulsed electric fields to disrupt bacterial biofilms.

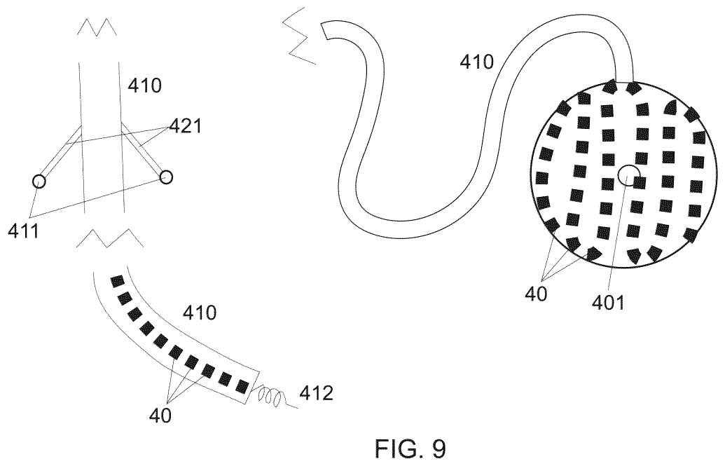

FIG. 9 provides a detailed view of the ICD or pacemaker system shown in FIG. 8, focusing on the electroporation-enabled generator, flexible lead structure, and tripod-like stabilization mechanism designed to secure the lead within the heart.

The upper section of FIG. 9 illustrates the implantable generator 400, which features multiple embedded electrodes 40 on its outer surface. These electrodes can deliver electroporation pulses to disrupt microbial biofilms and prevent infection at the implantation site. An access port 401 is included for external power connection, enabling electroporation energy delivery on demand or facilitating inductive recharging of the device's internal battery.

Extending from the generator is the lead 410, which is designed for both pacing and electroporation functions. The lead 410 is flexible, allowing it to adapt to patient movement while maintaining reliable electrical connections. Multiple electrodes 40 are embedded along its length, enabling targeted electroporation to prevent bacterial colonization along the lead body.

The lower section of FIG. 9 highlights the tripod-like stabilization mechanism 421, which consists of multiple support legs designed to anchor the lead 410 within the heart. At fixation points 411, pacing electrodes are positioned to deliver electrical stimulation to the heart. These pacing electrodes ensure effective signal transmission for cardiac rhythm management while the tripod-like legs provide mechanical stability, preventing lead migration due to cardiac motion or blood flow.

Additionally, electrode 412 is a screw-in fixation electrode embedded into the septal wall. This screw-in mechanism ensures continuous contact with the cardiac tissue, improving pacing stability and reducing the risk of lead dislodgement. The combination of screw-in fixation and tripod-like anchoring ensures that pacing electrodes 411 and 412 remain in optimal positions for sustained cardiac stimulation.

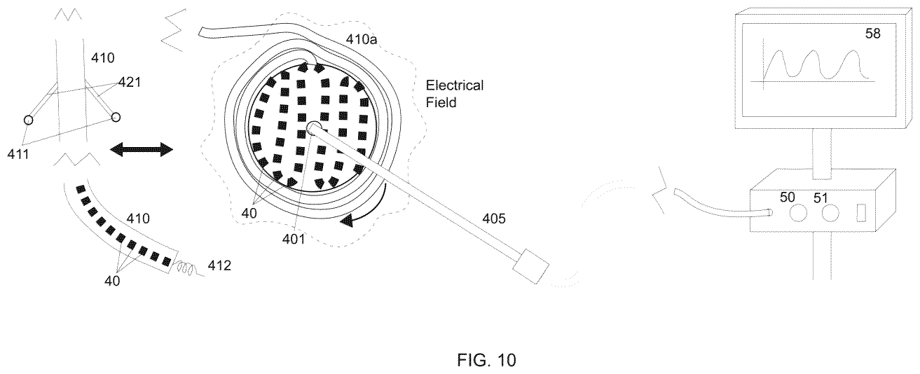

FIG. 10 illustrates an advanced implantable cardioverter defibrillator (ICD) or pacemaker system designed to integrate electroporation functionality for infection prevention. This system includes an external power interface that enables controlled electroporation pulses without relying solely on an internal power source. The figure highlights the external energy delivery mechanism, electroporation field generation, and structural stabilization of the lead system within the heart.

The system includes a power generator 50 with optional impedance module 51 and a monitoring/control system 58. This external system provides energy for electroporation, allowing healthcare providers to activate or adjust electroporation pulses in response to clinical needs. The monitoring system 58 provides real-time feedback on electroporation parameters, ensuring precise and controlled energy delivery. This setup allows for on-demand or scheduled electroporation to prevent biofilm formation and microbial colonization on the implanted components. By incorporating an external energy source, this system minimizes the power burden on the implanted device, reducing battery depletion while enabling high-energy electroporation cycles as needed.

The implantable generator features an access port 401 for external power connection. A percutaneous probe 405 interfaces with this port, allowing for controlled energy transfer to power the electroporation electrodes 40 embedded within the generator housing. This direct or inductive energy transfer enables precise electroporation activation without requiring an implanted high-energy power source. Surrounding the generator, the lead 410a is coiled externally, ensuring strain relief and preventing mechanical damage while also integrating electrodes 40 for extended electroporation field distribution. The arrows around the device indicate the spread of the electroporation field, which disrupts bacterial biofilms and prevents infection at the implantation site.

Lead 410 extends from the generator into the vascular system, reaching key locations within the heart. Along its length, multiple embedded electrodes 40 generate localized electroporation fields to prevent bacterial accumulation. At a critical anchoring point, a tripod-like stabilization mechanism 421 is deployed to secure the lead within the heart, reducing the risk of migration due to cardiac motion or hemodynamic forces. This stabilization structure ensures consistent lead positioning, improving both pacing effectiveness and electroporation efficiency.

At the fixation points of the stabilization mechanism, pacing electrodes 411 are strategically placed to provide continuous cardiac stimulation. Additionally, the lead terminates at electrode 412, which is a screw-in fixation electrode embedded into the septal wall. This screw-in mechanism enhances stability by anchoring the lead directly into the myocardium, preventing dislodgement and ensuring sustained pacing function. The combination of a screw-in electrode and tripod-like stabilization structure significantly enhances lead security while enabling precise electroporation field application around key contact points.

This system applies targeted electroporation at multiple levels to enhance infection prevention. The electrodes 40 embedded within the generator create an electroporation field around the device pocket, preventing bacterial colonization in the surrounding tissue. Additional electrodes along the lead 410 generate localized electroporation pulses to disrupt microbial biofilms that may form along the lead pathway. At the lead anchoring site, electroporation pulses are applied to ensure that no bacterial growth occurs around the tripod stabilization legs 421 or screw-in electrode 412, further reducing infection risks at critical contact points. Electrodes 40 may also comprise sensing mechanisms to detect properties such as pressure, microbial presence, biofilm formation, impedance variations, pH changes, or other biochemical markers indicative of infection or tissue response.

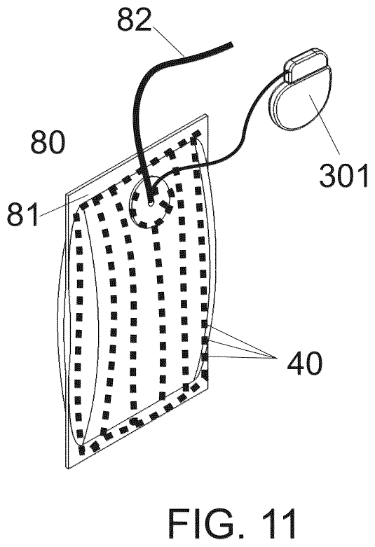

FIG. 11 illustrates a large drug reservoir system 80 designed for controlled drug storage and delivery. This system incorporates electroporation-enabled electrodes 40, a storage pouch 81, and a delivery catheter 82 for precise drug administration. The system may be powered by an implantable generator, which could be a power source, pacemaker, or implantable cardioverter defibrillator (ICD) 301. The generator 301 may also house additional electrodes (not shown) to facilitate pacing, defibrillation, or additional electroporation-based therapy.

The drug reservoir 80 features a flexible storage pouch 81 that holds liquid medication for sustained or controlled release. The pouch is constructed from a biocompatible material that ensures long-term stability while maintaining drug integrity. A delivery catheter 82 extends from the pouch, allowing for targeted drug administration into the patient's circulatory system, tissue, or another intended site. The catheter may include integrated flow control mechanisms, such as valves or micropumps, to regulate drug release based on real-time physiological feedback or pre-programmed dosing schedules.

Electrodes 40 are embedded along the walls of the reservoir, forming a patterned distribution that enables electroporation-based drug delivery. These electrodes can generate an electrical field that transiently increases cell membrane permeability, enhancing drug absorption and penetration into the target tissue. The electroporation function may be activated automatically or manually, based on clinical needs. The generator 301 supplies power to the electrodes and may also communicate with external monitoring devices to optimize electroporation parameters such as voltage, pulse duration, and frequency.

The power generator 301, which could be an ICD, pacemaker, or standalone power unit, is connected to the reservoir system. This integration allows for synchronized therapy delivery, where electroporation can be used to enhance drug uptake while the pacemaker or ICD manages cardiac function. Additionally, the power generator may feature its own set of electrodes (not shown), which could be used for pacing, defibrillation, or secondary electroporation applications.

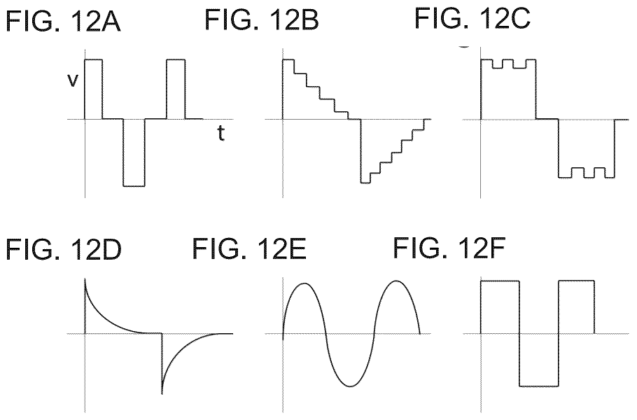

FIGS. 12A-12F illustrate six distinct waveform types that may be used for electroporation, each designed to optimize bacterial membrane disruption, enhance drug delivery, or provide controlled electrical stimulation. These waveforms vary in shape, polarity, and intensity, allowing for customization based on the desired biological effect. The waveforms in FIGS. 12A-12F are primarily biphasic, meaning they alternate between positive and negative voltages to prevent electrode degradation and maintain balanced charge distribution in the tissue.

FIG. 12A Biphasic Rectangular Pulses—This waveform consists of alternating rectangular pulses of equal amplitude and duration in both positive and negative directions. The biphasic nature ensures a balanced electrical field, minimizing residual charge buildup at the electrodes. This waveform is highly effective for reversible or irreversible electroporation, as the sharp transitions in voltage rapidly open pores in cell membranes.

FIG. 12B Stepped Waveform—The stepped waveform consists of incremental voltage steps that gradually increase and decrease in a controlled manner. This gradual transition in voltage intensity allows for finer control over membrane poration and may help reduce excessive tissue damage. Because the waveform is symmetric, it maintains an equal balance of positive and negative pulses, preventing excessive charge accumulation at the electrode sites.

FIG. 12C Mixed Rectangular Pulses with High-Frequency Modulation—This waveform combines high-amplitude rectangular pulses with smaller, high-frequency oscillations superimposed on each step. The combination of large and small pulses allows for strong membrane permeabilization while also modulating the electroporation effect. The symmetric nature of this waveform ensures that energy is distributed evenly across both positive and negative phases, reducing the likelihood of tissue polarization.

FIG. 12D Exponential Decay Pulse—An exponential decay waveform starts with a high initial voltage and gradually decreases over time. This waveform is useful for controlled electroporation because the rapid initial energy application ensures effective cell poration while the gradual decay minimizes excessive heating. The symmetry in this waveform ensures that tissue exposure to positive and negative charges remains balanced.

FIG. 12E Sinusoidal Waveform—This waveform consists of smooth, oscillating sinusoidal pulses that alternate between positive and negative voltages. The continuous nature of the waveform may help reduce abrupt electrical shocks to cells, allowing for a more gradual and controlled electroporation process. The symmetric pattern ensures that equal amounts of energy are delivered in both polarities.

FIG. 12F Bipolar Square Wave—The bipolar square wave is a high-amplitude, alternating waveform with sharp transitions between voltage levels. The sudden shifts between positive and negative voltages maximize electroporation efficiency by rapidly opening cell membranes. Because this waveform is balanced, it prevents excessive charge accumulation and reduces long-term electrode degradation.

The waveforms in FIGS. 12A-12F provide a diverse range of electroporation techniques, each with specific applications in bacterial biofilm disruption, drug delivery, and tissue ablation. The biphasic nature of these waveforms ensures charge neutrality, which helps prevent long-term electrode degradation and reduces tissue damage. The ability to control pulse duration, frequency, and intensity allows for precise energy delivery tailored to different medical applications.

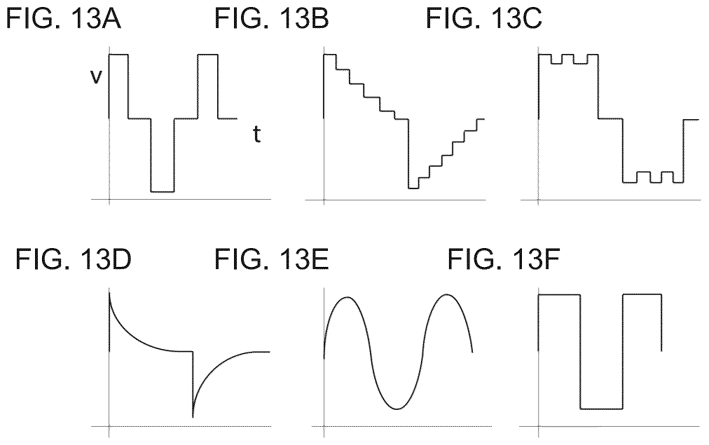

FIGS. 13A-13F illustrate six distinct waveform types similar to those in FIGS. 12A-12F but with a key difference: the waveforms in FIGS. 13A-13F may be asymmetric, monophasic, or biased towards one polarity. Unlike the balanced biphasic waveforms in FIGS. 12A-12F, these waveforms may have greater positive or negative charge accumulation, which can influence electroporation efficiency, bacterial disruption, or tissue response. The asymmetry or polarity bias can be useful for specific applications, such as directional electroporation effects or targeted cell membrane poration.

The waveforms in FIGS. 13A-13F introduce asymmetry, polarity bias, and unbalanced charge distributions, which can be useful for specific electroporation applications. Unlike the biphasic and charge-balanced waveforms in FIGS. 12A-12F, these waveforms can be tailored for directional electroporation, selective bacterial disruption, or controlled tissue targeting. While these adjustments can improve energy efficiency and treatment specificity, monophasic or polarity-skewed waveforms may lead to increased charge buildup at the electrodes, requiring careful monitoring to prevent long-term tissue damage or electrode degradation.

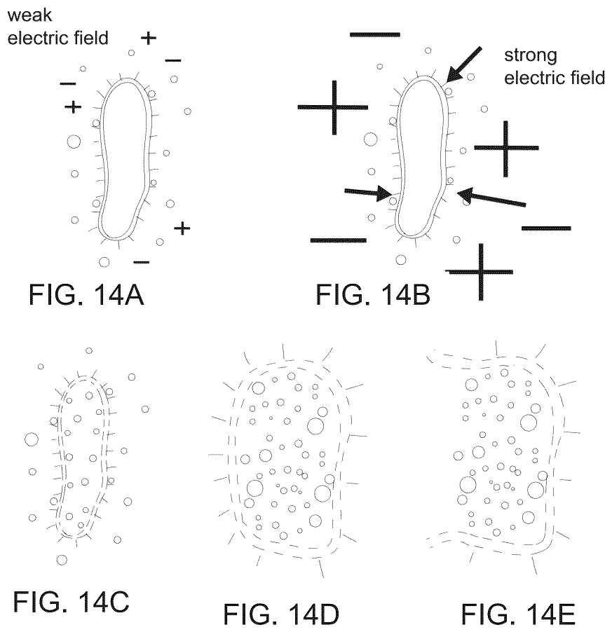

FIGS. 14A-14E show a simplified representation of the effects of electroporation on a bacterial cell, illustrating how different electric field strengths influence membrane permeability and lead to bacterial disruption. While this figure effectively demonstrates the key stages of electroporation, in reality, the process is more complex, involving uneven pore formation, intracellular effects, and dynamic membrane responses that are not fully depicted here.

In FIG. 14A, a weak electric field exists around the bacterial cell. Small positive and negative charges accumulate near the cell membrane, but the field strength is not sufficient to induce significant electroporation. In reality, even weak fields may cause minor transmembrane voltage changes, potentially leading to small, transient pores. However, in most cases, the bacterial membrane remains intact, and normal cellular function continues. This stage is typically associated with sub-threshold electroporation, where no lasting permeability changes occur.

FIG. 14B illustrates a bacterial cell subjected to a stronger electric field, resulting in high transmembrane voltage that stresses the lipid bilayer. The large positive and negative charges exert force on the membrane, leading to localized regions of high tension (indicated by arrows). This representation simplifies the actual process, as in reality, pores do not form uniformly around the membrane. Instead, pores tend to develop at the poles of the cell, where the electric field gradient is strongest. The degree of electroporation depends on field strength, pulse duration, and bacterial cell size and shape, factors that influence whether pores remain reversible or become permanent.

FIG. 14C shows the initial formation of nanopores across the bacterial membrane. These pores allow small molecules, such as ions or antibiotics, to pass through the membrane, disrupting homeostasis. If the electric field is removed at this stage, the membrane may reseal, a process known as reversible electroporation. In real scenarios, pore size and distribution vary depending on membrane composition, cell wall thickness, and applied pulse parameters.

FIG. 14D represents a stage where pore expansion leads to significant membrane disruption. Water influx causes swelling and osmotic imbalance, and the bacterial cell struggles to maintain internal stability. This depiction simplifies the actual biomechanical response of bacterial cells, which may include cellular repair mechanisms, enzyme activation, or DNA damage due to the strong electrical field. At this point, irreversible electroporation is likely occurring, meaning the bacterial membrane can no longer return to its original state.

In FIG. 14E, the bacterial cell has undergone catastrophic membrane failure, leading to cell lysis and death. This stage represents the outcome of high-intensity electroporation, where pore formation is so extensive that the bacterial cytoplasm leaks out. While the figure depicts a uniform breakdown, bacterial death may not be immediate. Some bacteria may enter a sublethal state, where structural damage has occurred, but repair is still possible under certain conditions. However, if enough damage accumulates, the bacterial cell cannot recover, leading to permanent eradication.

In accordance with examples of the present disclosure, the optimization of electroporation parameters—voltage, pulse duration, frequency, and waveform—may be meticulously engineered to target specific pathogens that contribute to infections in catheters, including PICCs and peripheral IVs, as well as conditions such as endocarditis. In some examples, parameters are distinctly tailored to address the unique cellular compositions of Gram-positive versus Gram-negative bacteria, acknowledging their differential resistance to electrical disruption due to variations in cell wall structure.

Further examples incorporate an adaptive control mechanism. This mechanism is designed to dynamically adjust the electroporation parameters in response to real-time feedback from integrated sensors that assess the impact on both bacterial cells and surrounding host tissues. Such adaptability ensures the precision in targeting pathogens while maintaining the integrity of nearby healthy tissues, especially critical in sensitive areas. In particular examples, the device includes real-time monitoring capabilities to detect and adjust the electrical parameters instantaneously, ensuring that the applied electroporation remains within safe limits for the surrounding healthy tissues. Furthermore, the long-term efficacy and stability of the electroporation effect in clinical settings are addressed through the incorporation of durability testing and feedback mechanisms. These features allow for continuous assessment and adjustment of treatment parameters to maintain optimal performance over time, demonstrating a commitment to patient safety and treatment effectiveness in diverse clinical environments.

Moreover, in certain examples, safety thresholds for electroporation parameters are established. These thresholds are rigorously defined through empirical studies to delineate the boundary conditions under which electroporation can be conducted without inducing adverse effects on host tissues. The versatility of this approach is underscored by its capability to be programmed with a diverse array of parameter settings, thereby accommodating a broad spectrum of infection types and patient-specific considerations. This level of customization and safety highlights the novel contribution of the disclosed systems and methods to the field of medical device technology, offering a significant advancement in the management and treatment of catheter-related infections and endocarditis.

In these or other examples, the catheter or member may be imbued with coatings that may prevent or induce clotting, or other coatings that aid in prolonging efficiency, such as but not limited to materials or compounds to promote sterility. For example, in certain examples, the device can be configured to work in synergy with antibiotic treatments, where electroporation-induced permeabilization of bacterial cells enhances antibiotic uptake, thereby overcoming resistance mechanisms. Additionally, the device may incorporate or be used in conjunction with antiseptic coatings or biofilm-disruptive agents applied to catheter surfaces, further reducing infection risks. These combined approaches not only leverage the direct antimicrobial effects of electroporation but also harness complementary mechanisms of action, offering a multifaceted strategy against infections associated with medical devices.

In one example, an implantable device comprises a plurality of electrodes configured for delivering pulsed electrical energy. At least one of these electrodes is coated with a catalytic material, such as platinum or a platinum alloy, deposited using techniques such as sputtering or electrochemical deposition. The catalytic coating is optimized to promote the two-electron reduction of dissolved oxygen in bodily fluids:

O₂+2H⁺+2e⁻→H₂O₂

When a predetermined cathodic potential is applied, the electrode generates H₂O₂ in situ, which diffuses locally to disrupt the bacterial biofilm matrix. The electrode design ensures that the H₂O₂ generation is confined to the vicinity of the electrode, thereby limiting systemic exposure.

Alternatively or in addition, the electrode may be coated with an enzyme-mimetic (nanozyme) material. Suitable materials include cerium oxide nanoparticles, manganese oxide nanostructures, or other compounds that exhibit oxidase-like activity. The enzyme-mimetic coating is applied via a polymer binder that adheres to the electrode surface, ensuring long-term stability in the physiological environment. Upon application of a cathodic potential, the nanozyme catalyzes the reduction of oxygen to H₂O₂, similarly to natural oxidase enzymes, but with improved stability and durability over prolonged implantation periods.

The catalytic or enzyme-mimetic coating is applied to achieve a thickness sufficient to catalyze the desired reaction without impairing the electrode's conductivity. Methods such as layer-by-layer deposition or dip-coating may be used to achieve uniform coverage. The electrical parameters (voltage, pulse duration, frequency) are optimized to balance effective H₂O₂ generation with the minimization of collateral tissue damage. In one example, high-voltage, microsecond-scale pulses may be applied periodically during treatment sessions under controlled conditions (e.g., sedation). The electrodes may be arranged in an alternating pattern or as bipolar pairs along the device, allowing for targeted application of the electric field. Wiring may be grouped by polarity to simplify the circuitry while ensuring that the energy delivery is focused at the coated electrode surfaces.

The systems and methods herein may be particularly suited for implantable devices that are susceptible to biofilm-related infections, including but not limited to pacemaker leads, defibrillator leads, and vascular catheters. By enhancing localized H₂O₂ production, the device provides an adjunct or alternative to systemic antibiotic therapy, potentially reducing the incidence of device-related infections.

The application of electroporation in medical devices extends beyond catheters and drug reservoirs, offering novel infection control solutions for orthopedic implants, hemodialysis ports, neural interfaces, vascular stents, and gastrointestinal devices. These implementations leverage controlled pulsed electrical fields to disrupt bacterial cell membranes, thereby preventing infections, extending device longevity, and enhancing therapeutic outcomes.

Orthopedic implants such as titanium plates, screws, and joint prostheses present a significant infection risk due to their direct interface with bone and soft tissue. Electroporation-enabled implants may integrate microelectrodes on their surfaces, delivering periodic electrical pulses to prevent bacterial colonization and biofilm formation. The electrode surfaces can be arranged in a grid-like pattern to ensure uniform coverage, and an embedded power interface may allow for intermittent external activation. Inductive coupling or a low-profile external energy delivery mechanism could provide periodic power to the implant without requiring internal batteries. The optimal waveform for orthopedic applications may include biphasic pulses to reduce electrode degradation and minimize tissue heating.

Long-term venous access for dialysis patients is associated with a high risk of bloodstream infections. Embedding electrodes around the catheter cuff could periodically sterilize the port, reducing bacterial load and preventing systemic infections. A ring electrode design at the catheter entry point could be automatically triggered based on impedance monitoring to detect early bacterial growth. Power requirements for this system may be addressed via intermittent connection to an external power source at the dialysis clinic, reducing the need for an implanted battery. Waveform selection may include high-frequency short bursts to maximize bacterial disruption while preventing clot formation.

Implantable drug pumps for chronic conditions such as Parkinson's disease or diabetes could incorporate electroporation electrodes near the catheter tip to enhance localized drug absorption and prevent infection at the injection site. By applying controlled pulsed fields, cell permeability can be temporarily increased to improve drug uptake. These electrodes may be powered by a rechargeable battery or inductive charging mechanism, activated only during drug administration. Square wave pulses may provide the most controlled electroporation effect in these applications, ensuring precision in drug delivery.

Neurostimulators implanted for pain management or spinal cord injuries could integrate electroporation to sterilize lead entry points and prevent bacterial colonization. Electrodes embedded within lead insulation may generate controlled electrical fields around the surgical insertion site. The system could be powered using an auxiliary rechargeable capacitor harvested from the existing neurostimulator battery. Adaptive pulse width modulation could be used to tailor electroporation intensity based on tissue impedance and infection risk, ensuring safety and efficacy.

Cochlear implants are susceptible to infections at the electrode-tissue interface. Integrating electroporation into the implant casing could provide a means of periodically sterilizing the surgical site. Miniaturized electrodes embedded along the implant housing may deliver targeted electrical pulses, disrupting bacterial cell membranes without interfering with auditory nerve stimulation. Power may be sourced from the existing implantable battery with software-controlled activation cycles. Low-intensity biphasic pulses may be preferred to prevent interference with neural stimulation.

Brain implants for epilepsy treatment, neuroprosthetics, or deep brain stimulation could benefit from electroporation-based infection control. Electrodes arranged in a ring pattern around the craniotomy site could periodically apply electrical pulses to prevent bacterial colonization. Power could be harvested from external transcranial stimulation or through wireless inductive charging. High-intensity, short bursts may provide the most effective bacterial disruption while preventing unintended neural interactions.

Infections in esophageal, biliary, or intestinal stents can cause severe complications. Conductive coatings or integrated microelectrodes along the stent's inner surface could provide periodic electroporation-based bacterial control. These electrodes could be activated via an external transdermal energy transfer unit, allowing patients to receive antimicrobial treatments without invasive intervention. Medium-voltage, high-frequency pulses may be optimal for disrupting bacterial biofilms without affecting the surrounding tissue integrity.

Vascular grafts and stents used in bypass surgeries or aneurysm repairs are prone to bacterial colonization and thrombosis. Integrating electroporation electrodes within the graft walls may allow for periodic sterilization of the lumen, reducing infection risks. The power supply may be delivered via catheter-based external activation or energy harvested from blood flow. Pulsed fields tailored to endothelial healing may be used to minimize restenosis risks while maintaining infection control.

In one example, an LVAD driveline is integrated with electroporation or pulsed field ablation (PFA) electrodes positioned along the exterior sheath of the cable to prevent bacterial colonization and biofilm formation at the percutaneous exit site. The electrodes may be embedded within the outer insulation layer of the driveline, e.g., arranged in a circumferential or spiral pattern, to ensure uniform electric field coverage. The system may be programmed to deliver periodic low-energy PFA pulses to disrupt bacterial membranes, mitigating infection risk while maintaining structural integrity of the driveline.

In another example, the PFA system is configured with real-time bacterial detection to provide targeted infection control. Embedded impedance or biofilm sensors may be placed near the skin exit site to monitor microbial colonization levels. When the sensor detects an increase in bacterial load or a shift in impedance indicative of biofilm formation, the system may automatically initiate a PFA cycle. The system can dynamically adjust the pulse parameters—such as frequency, duration, and amplitude-based on real-time feedback to optimize bacterial disruption while minimizing energy consumption and patient discomfort.

In another example, the PFA system is externally powered and controlled via a wearable interface. Patients or clinicians can manually activate PFA treatments when infection is suspected or detected through visual inspection, clinical symptoms, or microbiological testing. The wearable controller may include a rechargeable power source and a wireless communication link to adjust electroporation parameters remotely. This approach allows for non-invasive, on-demand treatment of LVAD driveline infections without the need for surgical intervention or systemic antibiotic administration.

In some examples, the driveline's percutaneous exit site is equipped with a dedicated electroporation-enabled cuff. This flexible, biocompatible cuff wraps around the exit site and delivers controlled PFA pulses to disrupt bacterial biofilms on the skin surface and surrounding tissue. The cuff may be designed as a replaceable or disposable component, allowing clinicians to swap out the electrode assembly periodically while maintaining the primary driveline in place. Additionally, the cuff may integrate antimicrobial coatings or drug-eluting materials to provide a dual-modality infection prevention strategy, combining electroporation with chemical antimicrobial effects.

In another example, the LVAD driveline may incorporate capacitive coupling electrodes embedded within its structure to provide non-contact electroporation treatment. These electrodes generate an oscillating electric field that penetrates surrounding tissue without requiring direct conductive contact, reducing the risk of electrode fouling or degradation over time. The capacitive coupling system may be programmed to deliver electroporation pulses in conjunction with patient movement or during scheduled treatment intervals to maximize its antimicrobial effects.

In one example, the LVAD controller is modified to include an electroporation module that synchronizes PFA pulses with the cardiac cycle. By timing electroporation treatment during specific phases of the cardiac cycle—such as diastole—the system can minimize interference with normal LVAD function while ensuring effective bacterial disruption. This synchronization may be achieved through integration with the LVAD's onboard sensors or external cardiac monitoring systems.

In another example, an implantable battery pack or energy harvesting unit is used to power the PFA system without requiring an external power source. The energy harvesting unit may derive power from the LVAD's existing electrical circuitry, fluid movement, or physiological motion. This approach enables continuous or periodic electroporation treatment without the need for patient intervention, enhancing long-term infection control in LVAD patients.

In yet another example, the PFA system is designed for combination therapy, where electroporation pulses are delivered in conjunction with localized antibiotic infusion. The driveline may feature an integrated drug-delivery lumen or microfluidic channels that allow small volumes of antibiotics or antimicrobial agents to be delivered directly to the exit site. When electroporation pulses are applied, bacterial cell membranes become temporarily permeabilized, enhancing antibiotic penetration into biofilms and increasing the overall efficacy of antimicrobial treatment.

By integrating PFA-based infection control into LVAD drivelines, these examples aim to address the most significant complication associated with long-term LVAD support—persistent and recurrent driveline infections. The disclosed systems provide a non-thermal, non-chemical sterilization approach that reduces the need for systemic antibiotics, surgical debridement, or LVAD replacement due to infection-related complications.

Continuous glucose monitors, pH sensors, or implantable biochemical sensors often require long-term implantation, making infection control critical. Miniaturized electrodes integrated around the sensor tip could provide electroporation-based sterilization, extending sensor lifespan. A small rechargeable battery or passive energy-harvesting mechanism could power these pulses. Low-energy biphasic pulses may be most effective to ensure minimal interference with sensor function.

Future electroporation-enabled devices may leverage energy-harvesting techniques such as piezoelectric or thermoelectric conversion to reduce battery dependency. AI-driven sensor fusion could predict infection risks based on physiological markers and autonomously trigger sterilization cycles. These adaptive systems could optimize waveform selection, adjusting pulse intensity and frequency in real-time to minimize tissue damage while maximizing bacterial eradication.

Wireless data transmission and remote monitoring could allow clinicians to track electroporation treatment efficacy and adjust parameters as needed. Devices could integrate real-time feedback systems, logging electroporation events, sensor data, and bacterial load trends to refine treatment protocols. Secure data encryption and integration with electronic health records may further enhance patient management and compliance.

Electroporation effectiveness can be improved through real-time waveform adjustments based on impedance changes or tissue response. Dynamic pulse modulation may allow devices to optimize electroporation for different tissue types, enhancing both safety and efficacy. For example, higher frequency pulses may be applied for biofilm disruption, while lower frequencies could target deep-seated bacterial infections.

By integrating these novel electroporation-based infection control methods into a wide range of medical devices, the risk of device-related infections can be significantly reduced. These advancements may improve patient outcomes, extend device functionality, and provide safer, longer-lasting therapeutic solutions.

In one example, an implantable diuretic delivery device (“the device”) includes a reservoir to contain one or more diuretic agents (e.g., loop diuretics) suitable for treating fluid retention in heart failure patients. The reservoir is formed from a biocompatible material capable of maintaining drug stability over extended periods. An infusion assembly (such as a pump or flow regulator) regulates the release of diuretic into the patient's vasculature or peritoneal cavity, thereby helping to manage fluid overload. Positioned on or adjacent to the device is a refillable access port that sits just beneath the patient's skin. This port permits clinicians to percutaneously inject additional diuretic solution using a needle or specialized connector at predefined intervals, obviating the need for frequent external injections or oral dosing regimens.

The system further comprises a PFA module that generates controlled pulses of high-voltage electrical energy. One or more electrodes or conductive surfaces are arranged on or within the device's port, reservoir housing, or associated catheter. These electrodes deliver electroporation pulses to a localized region surrounding the device, including interior surfaces where microbial colonization could occur. A microcontroller or pulse generator within the device drives short, high-voltage pulses (e.g., microseconds to milliseconds in duration). The electrodes may be embedded along the interior surfaces of the fluid reservoir, positioned around the catheter's inner lumen, or arranged near the refill port to specifically target areas prone to infection. The device may automatically deliver PFA at scheduled intervals (e.g., once daily or weekly) or upon detecting early signs of bacterial growth, using sensor data such as impedance or temperature increases. Alternatively, clinicians may trigger an on-demand PFA cycle via a wireless or direct interface if an infection is suspected.

To facilitate both drug refill and potential PFA power delivery, the device includes one or more access mechanisms. A needle or cannula can be inserted into the refill port under sterile conditions, enabling both (i) transfer of diuretic solution into the reservoir and (ii) optional electrical coupling to the PFA module if additional power or reprogramming is required. In some examples, the device may contain an inductive coil or antenna to receive power wirelessly. This power can be used to recharge an internal battery or capacitor bank that stores the energy for delivering high-voltage pulses without relying solely on the implant's primary battery. The port incorporates sealing structures that maintain sterility when not accessed for refilling. If an electrical connection is involved, conduction pathways are shielded to prevent fluid ingress and ensure patient safety.

To optimize diuretic delivery, the device may include one or more sensors that measure relevant physiological parameters. Sensors may monitor central venous pressure, pulmonary artery pressure, or peritoneal fluid buildup to inform dosing adjustments. Alternatively or in addition, the sensors may estimate intravascular or intraperitoneal fluid status to regulate infusion rates. Temperature, impedance, or chemical sensors may detect early microbial activity. When triggered, the device can initiate a PFA sequence to eradicate or reduce biofilm formation. A microcontroller in communication with these sensors can adapt the diuretic dosing schedule based on fluid overload thresholds while also managing PFA delivery intervals. Clinicians can access these sensor readings through a wireless interface or direct port-based data download, facilitating personalized patient management.

The device is surgically implanted, positioning the reservoir and pump subcutaneously or submuscularly. The refill port is placed in an accessible location for percutaneous needle access. A clinician or technician configures dose rates, refill intervals, and the default PFA schedule. If wireless charging is used, the external coil or charger is demonstrated to the patient for at-home or clinic-based recharging sessions. The implanted pump dispenses medication according to programmed schedules or real-time sensor data, maintaining stable fluid balance. At preset intervals, the patient visits a healthcare provider who refills the reservoir by inserting a sterile needle through the port. The system periodically or on-demand delivers high-voltage pulses through the port or catheter electrodes, disrupting microbial cells and reducing infection risk. Sensor readings (pressure, volume, infection markers) are continuously or intermittently monitored. Clinicians can adjust medication dosage or PFA timing if data suggest an increased infection risk or changes in the patient's fluid status.

Integrated PFA can periodically cleanse the reservoir and associated fluid pathways, minimizing the formation of dangerous biofilms that often lead to device failure or sepsis. By maintaining appropriate diuretic therapy and infection-free operation, the device may reduce the frequency of acute heart failure exacerbations and subsequent readmissions. The implant's refill port allows for quick outpatient medication replenishment, replacing frequent or high-dose oral diuretic regimens that can be less predictable. Whether through direct needle-based power connection or wireless inductive coupling, the system remains flexible for various clinical settings and patient compliance levels. Additional therapeutic agents (antibiotics, vasodilators) may be loaded into the reservoir for combination therapy. The implant could communicate wirelessly with a patient's smartphone or physician's system, sending alerts if sensor data show rising infection potential or if reservoir levels are low. Different pulse waveforms (biphasic, multi-pulse) can be programmed to enhance electroporation efficacy while minimizing damage to surrounding tissues.

In one example, a peripherally inserted central catheter (PICC) is modified or constructed to include one or more electrically conductive elements (e.g., wires, bands, or printed circuits) along the catheter's length. These elements act as PFA electrodes, configured to deliver controlled pulses of electrical energy sufficient to induce electroporation in microbial cells that may colonize the internal or external catheter surface.

A pulse-generating module, either external or integrated into a hub near the catheter's proximal end, supplies intermittent high-voltage pulses. Through carefully selected voltage levels, pulse widths, and repetition rates, the system achieves localized pulsed field ablation of pathogens without causing clinically significant thermal damage to blood cells or vessel walls.

The catheter body is formed of a flexible, biocompatible material, such as polyurethane, silicone, or PTFE. Sized to fit peripheral veins in the arm and advance into central venous circulation. Thin, conductive metal filaments embedded within the catheter wall, running longitudinally. Metallic or conductive polymer layers deposited or printed on the catheter's external or internal surfaces. Proximal end of each electrode is connected to an electrical hub or connector for coupling to a pulse generator. The catheter hub can house electrical contacts that mate with an external cable or module, which optionally includes a luer lock or similar fitting for infusion of medications, fluids, and flush solutions.

An external pulse generator may include a bedside or portable device capable of generating short, high-voltage pulses (e.g., 50-3,000 V) with microsecond to millisecond durations. This generator may feature user controls or programmable logic for scheduling PFA treatments (e.g., once per day, once per week) or delivering pulses on demand when an infection is suspected. The system can deliver routine prophylactic pulses to preempt biofilm formation. A feedback sensor (e.g., impedance or temperature sensor) can detect changes indicative of microbial growth, triggering or alerting clinicians to perform a targeted PFA cycle. For safety, current-limiting circuits ensure that only minimal safe currents traverse the bloodstream. Pulse shaping or gating circuitry prevents accidental prolonged high-voltage exposure, reducing the risk of vessel or tissue damage.

The PICC is placed using standard sterile technique, typically via ultrasound guidance. Once secured, the catheter hub is connected to standard IV lines or medication infusion systems. The external pulse generator is connected to the catheter's electrical contacts (or wirelessly powered in certain examples). Pulse parameters (voltage, duration, frequency) are preset or dynamically adjusted by a clinician or control algorithm. At predetermined intervals, low-level pulses are delivered to disrupt early biofilm formation on or within the catheter. If logs or sensor data indicate minimal microbial presence, these pulses may be spaced out or reduced in intensity. Should infection be suspected (e.g., local redness, fever, positive blood cultures), the system can be activated for a more aggressive or higher-intensity pulsed field cycle. Concurrent antibiotic therapy may be administered through the catheter to leverage electroporation-enhanced antimicrobial penetration. Clinicians can monitor catheter patency, patient status, and sensor readings. Standard PICC care protocols (e.g., flushing with saline, dressing changes) are supplemented by periodic PFA treatments.

Advantages include: reduced infection rates, as pulsed fields disrupt biofilms and microbial communities, potentially lowering CRBSIs and associated healthcare costs; extended catheter longevity, by preventing or resolving early-stage colonization, the PICC may remain in use longer, reducing the need for replacement procedures; enhanced antibiotic efficacy, since temporary pores formed by electroporation can improve antibiotic diffusion into microbes and biofilms; minimal thermal damage, as electroporation is largely non-thermal, minimizing heat injury to blood cells or vascular endothelium; and integration feasibility, as the electrodes and connectors can be incorporated into conventional PICC designs with only modest increases in complexity and diameter. A small battery may be integrated into the catheter's hub to deliver pulses for short durations, replenished or swapped periodically. Inductive coils near the catheter hub could receive power from an external source, eliminating the need for external cables. Certain designs may integrate both a primary infusion lumen for fluids/medications and a separate lumen housing electrodes and sensors.

The systems and methods herein may address a longstanding challenge in PICC-based venous access: catheter-related infections arising from biofilm formation. By integrating pulsed field ablation capabilities directly into the PICC structure, clinicians can proactively sterilize the device lumen and surrounding tissue environment. This approach has the potential to significantly reduce infection rates, prolong catheter dwell time, and improve patient safety and comfort compared to standard PICC lines.

In one example, the implantable device may comprise a biocompatible housing constructed from materials such as titanium, implantable-grade steel, or medical-grade polymers. The housing may enclose one or more reservoirs, each capable of holding a specific drug formulation. The device may be designed in various shapes or sizes, selected according to the intended anatomical site of implantation and patient requirements. For instance, a flattened, contoured form factor may be employed for subcutaneous placement in the chest wall, while a more rounded or elongated configuration may be suitable for abdominal implantation.

In another example, the reservoirs may be flexible pouches made from inert polymeric membranes. These membranes may be selected to ensure long-term stability of the stored drugs. Alternatively, the reservoirs may comprise rigid chambers with controlled release mechanisms. The device may also include mechanical or microfluidic pumps, valves, or diffusion membranes configured to govern drug release rates precisely.

In one example, the device may include multiple reservoirs, each holding a distinct therapeutic agent. A set of micro-pumps may draw fluid from each reservoir and deliver the drug via an outlet catheter or port into the patient's circulation or targeted tissue space. In some examples, the device may include peristaltic pumps, piezoelectric pumps, or osmotic pumps, any of which may be miniaturized and low-power. Such pumps may be operated by the device's processor according to pre-programmed schedules or in response to sensed physiological parameters.

Alternatively, in one example, the device may rely on controlled-release membranes that allow passive diffusion of the drug at a stable rate. By applying adjustable pressure or mild electrical stimulation, the device may modulate membrane permeability to achieve dynamic dosing. Another approach may involve hydrogel-based systems that swell or contract in response to electrical stimulation, thereby adjusting drug release rates.

In one example, the device may include an externally accessible refill port, located beneath the skin, identifiable by palpation, imaging, or built-in markers. The port may feature a self-sealing silicone septum that allows for repeated needle insertion without significant degradation. This configuration may enable healthcare providers to refill the reservoirs periodically using a minimally invasive transcutaneous approach.

In one example, the device may incorporate optical or magnetic guidance systems to aid in needle alignment, ensuring accurate penetration into the correct reservoir channel. The port may also be equipped with internal valves allowing selective refilling of different reservoirs through a single access point. This versatile refill architecture may simplify maintenance, extend device lifespan, and reduce the need for surgical interventions to replace or replenish medications.

To address the risk of infection commonly associated with implantable devices and transcutaneous ports, the present disclosure contemplates integrating an electroporation or PFA assembly. In one example, this assembly may include multiple electrodes arranged around the port or along fluid conduits. The device's processor may deliver short, high-voltage pulses, creating intense electrical fields that disrupt bacterial cell membranes.

In one example, the device may apply electroporation cycles during or immediately following a refill procedure, minimizing the likelihood of bacterial introduction. Additionally, in some examples, sensors capable of detecting microbial load or changes in fluid properties may trigger unscheduled electroporation cycles. For instance, if an increase in microbial presence is detected, the device may initiate a sterilization protocol to neutralize bacterial contamination.

In one example, the device may employ pulsed electric fields with field strengths ranging from approximately 500 V/cm to about 2000 V/cm. Such intensities may be sufficient to induce cell membrane poration in bacterial cells without causing irreversible thermal damage to surrounding host tissues. In another example, the pulse duration may be optimized to the order of tens to hundreds of microseconds. For instance, a series of eight to ten pulses, each lasting around 100 microseconds, may achieve effective bacterial disruption while maintaining a safe thermal profile.

In one example, the pulse frequency could be tuned to optimize bacterial eradication while minimizing discomfort or collateral effects. Frequencies between 1 Hz and 10 Hz may provide enough time between pulses for the tissue and device surfaces to dissipate any minor heat accumulation. Alternatively, applying a burst of pulses at a higher frequency, such as 100 Hz, for a very short interval may quickly achieve the required electric field conditions before switching to a lower-frequency maintenance mode. The choice between a continuous low-frequency regimen and a high-frequency burst mode could be informed by computational modeling and in vitro experiments.

In another example, the electrode configuration may be arranged in a concentric pattern around the device's port. Multiple ring-shaped electrodes, each with a diameter of about 2-5 mm and spaced 1-3 mm apart, may generate relatively uniform fields around the port surface. Alternatively, a pair of linear electrodes spaced 5-10 mm apart could produce a well-defined electric field gradient that may be rotated or shifted by activating different electrode pairs. In yet another example, a matrix of microelectrodes placed at intervals of 1-2 mm across the surface of the device may allow for dynamic steering of the electric field. By selectively activating subsets of these electrodes, the device may focus the field in a particular region identified as having higher bacterial load.

In one example, the system for electroporation-based bacterial disruption may employ a monopolar configuration, wherein a single electrode acts as the active element while a return path is provided via a separate, larger grounding electrode positioned within or outside the device. This configuration simplifies wiring and allows for greater flexibility in electrode placement, particularly in catheter-based applications. In another example, a bipolar electrode configuration may be used, where two electrodes of opposite polarity are arranged in close proximity to generate a well-defined electric field. This approach ensures more localized bacterial disruption and reduces the overall energy required for effective electroporation.

In a further example, the device may utilize a multipolar or alternating polarity electrode arrangement, wherein multiple electrodes are placed in an array with alternating anodes and cathodes. This design ensures uniform field distribution along the treatment area, minimizing inconsistencies in bacterial disruption. The system may also incorporate a time-sharing or sequential polarity switching mechanism, wherein electrode polarities periodically reverse, thereby preventing excessive ion buildup and reducing the risk of electrochemical reactions that could degrade electrodes over time.

The system may further incorporate various electrical wiring configurations to optimize electroporation efficiency. In one example, electrodes and/or sensors may be connected in a parallel arrangement, wherein all electrodes of the same polarity are electrically coupled. This ensures uniform energy distribution across multiple electrodes, making it particularly useful for multi-lumen catheters or large-area electroporation applications. Alternatively, in another example, a series connection may be implemented, allowing current to flow sequentially through a set of electrodes. This approach enables a controlled voltage drop along the electrode path, optimizing field gradients while maintaining power efficiency. Electrodes may also be independently wired.

In some examples, an independent electrode control system may be employed, wherein each electrode is separately addressable via an active switching circuit. This configuration allows for real-time modulation of electrical parameters, dynamic field shaping, and targeted electroporation in specific areas of a catheter or implantable device. In one variation, an interdigitated electrode array may be implemented, where alternating positive and negative electrodes are arranged in a patterned configuration. This design enhances uniformity of the electric field and provides a distributed treatment effect, particularly in catheter lumens or fluid pathways.

To prevent electrical arcing, various strategies may be incorporated into the system design. In one example, pulse waveform optimization may be employed, wherein voltage is applied in controlled pulse shapes, such as square waves, exponential decay pulses, or biphasic pulses. This approach minimizes excessive charge buildup and reduces dielectric breakdown risks. In another example, electrode spacing optimization may be implemented, ensuring that the distance between electrodes is configured to balance field strength and arc prevention.

In some examples, dielectric barriers or insulating layers may be incorporated to prevent unintended current discharge. These barriers may be formed from biocompatible materials such as medical-grade silicone, ceramic coatings, or polymer-based insulating films. In another example, the system may include pulse timing and duty cycle control, wherein the duration and frequency of applied pulses are adjusted dynamically to prevent overheating and excessive ion accumulation.

Additionally, the device may integrate grounding and shielding mechanisms to further reduce the risk of unwanted electrical discharge. In one example, conductive shielding elements may be placed around electrode assemblies to confine the applied electric field to a defined treatment zone. In another example, an adaptive liquid conductivity regulation system may be employed, wherein the electrolyte concentration in the catheter lumen or surrounding treatment area is monitored and adjusted to maintain optimal electrical conductivity while preventing uncontrolled current flow.

In one example, magnetic fields could be incorporated to enhance bacterial disruption or to influence the orientation of certain microbial cells. A low-level alternating magnetic field on the order of a few millitesla (mT) could be applied before or after the electric pulse treatment. This might improve the overall sterilization process by agitating bacterial clusters and making them more susceptible to subsequent electric pulses.

In another example, the ratio of electric to magnetic field intensity could be adjusted based on the particular bacteria or biofilm characteristics. Computational modeling may reveal that a certain bacteria type requires a minimum electric field of roughly 800 V/cm and benefits from a weak oscillating magnetic field at 1-5 mT to disrupt stable biofilm structures. Initial in vitro testing may refine these values, and subsequent in vivo studies in animal models could confirm which parameter sets maximize bacterial kill rates while minimizing tissue irritation.

In one example, sensor feedback integrated into the device could dynamically adjust pulse parameters. If sensors detect bacterial metabolic markers at elevated levels, the system may increase pulse field strength from a baseline of 700 V/cm up to 1500 V/cm for a short treatment cycle. If sensors indicate potential tissue irritation, the device may shorten pulse duration from 100 microseconds to 50 microseconds or reduce pulse frequency from 10 Hz to 2 Hz. Over time, accumulated data on bacterial response rates, tissue conditions, and healing patterns may guide increasingly refined protocols.

In another example, preclinical studies may employ computational simulations using finite element models to predict electric field distribution within various tissue geometries. These models could incorporate realistic tissue conductivity values (e.g., 0.2-0.7 S/m) and electrode surface properties. By correlating simulation results with data from bench-top experiments on bacterial cultures grown on device-like surfaces, engineers and clinicians may converge on an optimal pulse regimen—for instance, eight pulses at 1000 V/cm, each 75 microseconds long at a frequency of 5 Hz. Such parameters may then be tested in vivo, starting with animal models, where tissue response and healing over several weeks or months could validate the safety and efficacy of the chosen parameters.

In one example, if thermal effects present a concern, infrared thermography or implanted temperature sensors may monitor local temperature changes. This feedback could prompt the device to switch to shorter pulses or lower field strengths if tissue temperature rises above a safe threshold (e.g., a 1-2° C. increase over baseline). Likewise, if bacterial reduction proves suboptimal, the device may increase pulse number or field strength in subsequent treatment sessions, guided by sensor data.

In another example, the device could include a manual override mode, enabling a physician to externally command higher-intensity treatments—e.g., a short burst of 2000 V/cm pulses at 10 Hz—if a severe infection is detected and immediate, aggressive action is required. Such a mode may be used infrequently and/or only after ensuring tissue integrity and patient comfort through imaging and diagnostic tests.

Through these iterative processes—computational simulations, lab-based optimization, animal studies, and eventually clinical trials—the parameters for using pulsed field energy to disrupt bacterial colonization may be steadily refined. Ultimately, the chosen parameters may balance efficacy in bacterial eradication with patient safety, minimal collateral damage, and comfort, resulting in a reliable protocol for long-term infection control in implantable devices.

Examples of electrical parameters for bacterial eradication are shown in the two tables below. The tables provide critical data supporting the optimization of electroporation parameters for bacterial eradication and biofilm disruption, directly informing the design and functionality of the disclosed device. By outlining the effects of varying voltage levels, pulse frequencies, pulse durations, and electrochemical stimulation on bacterial viability, the tables establish a technical framework for selecting electroporation settings that maximize antimicrobial efficacy while minimizing risks such as tissue damage or interference with implanted medical devices. This data ensures that the system can be precisely calibrated to disrupt bacterial membranes, enhance antibiotic penetration, and prevent biofilm formation, thereby improving infection control in catheters, implantable reservoirs, and other medical applications. Moreover, by identifying potential risks and considerations associated with different electrical parameters, the tables contribute to the development of safe, effective treatment protocols that optimize patient outcomes while maintaining device integrity.

TABLE 1

| Electrical Parameter | Expected Effect on Biofilm |
|---|---|
| Low-frequency AC (0.1-10 Hz, | May disrupt quorum sensing and |
| low voltage ~1 V/cm) | bacterial adhesion. |
| Medium-frequency AC (10-1000 | Can impair ion exchange in bacteria, |
| Hz, moderate voltage) | leading to metabolic disruption. |
| Pulsed High-Voltage DC | May induce electroporation in bacteria, |
| (PFA-like, microsecond | enhancing antibiotic penetration. |
| pulses) | |
| Electrochemical Effects | Could generate ROS (H₂O₂, |
| (Cathodic/Anodic Stimulation) | hydroxyl radicals) for antimicrobial |
| | action. |

TABLE 2

| | | | Potential Risks & |
|---|---|---|---|
| Parameter | Range/Types | Effect on Bacteria & Biofilm | Considerations |
| Voltage (V/cm) | Low (0.1-1 V/cm) | Disrupts quorum sensing, bacterial | Too low may not penetrate |
| | | adhesion, and early biofilm | mature biofilms. |
| | | formation. | |
| | Medium (1-10 | Impairs bacterial ion exchange, | May need prolonged |
| | V/cm) | reduces metabolism, and weakens the | exposure to be effective. |
| | | biofilm matrix. | |
| | High (10-100+ | Induces bacterial electroporation, | Risk of tissue damage, EMI |
| | V/cm) | membrane disruption, and enhances | with pacemaker function. |
| | | antibiotic penetration. | |
| Frequency (Hz) | Low (0.1-10 Hz) | Affects bacterial communication | Limited impact on mature |
| | | (quorum sensing), reduces adhesion | biofilms. |
| | | to surfaces. | |
| | Medium (10-1000 | Disrupts bacterial metabolic activity, | Could affect host cells with |
| | Hz) | can interfere with membrane ion | prolonged exposure. |
| | | transport. | |
| | High (>1000 Hz) | Increased electroporation likelihood, | High frequencies may |
| | | cell death due to energy | generate excess heat. |
| | | accumulation. | |
| Pulse Shape | Monophasic DC | Stronger impact on bacterial | Higher risk of local heating. |
| | pulses | membranes; can induce | |
| | | electroporation. | |
| | Biphasic | Reduces ionic buildup, prevents rapid | Requires careful tuning to |
| | (Alternating) | biofilm adaptation, and may help | avoid host cell damage. |
| | Pulses | prevent regrowth. | |
| | Trains of Pulses | May enhance bacterial susceptibility | Requires optimization of |
| | | over time, particularly for thick | duration and duty cycle. |
| | | biofilms. | |
| Pulse Duration | Short (≤10 μs) | Selectively affects bacteria due to | Needs higher voltage to be |
| (μs to ms) | | their higher membrane | effective. |
| | | resistance than mammalian cells. | |
| | Medium (10 μs- | Stronger biofilm penetration, | May still spare host tissue if |
| | 1 ms) | moderate risk to host cells. | tuned properly. |
| | Long (1 ms- | Greater biofilm matrix disruption, | Thermal effects and host |
| | seconds) | increased bacterial death, but higher | cell damage possible. |
| | | risk of collateral tissue effects. | |
| Electroporation | Reversible (sub- | Creates transient pores, allowing | Biofilm can recover if |
| Intensity | threshold) | antibiotics or immune cells to | insufficiently treated. |
| | | penetrate biofilms. | |
| | Irreversible | Leads to complete bacterial cell lysis, | May damage pacemaker |
| | (above threshold) | biofilm collapse. | leads or host cells. |
| Electric Field | Homogeneous | Ensures even bacterial exposure, | Hard to achieve in complex |
| Uniformity | field | potentially improving treatment | environments like |
| | | efficiency. | pacemaker leads. |
| | Heterogeneous | May selectively target bacterial | Risk of uneven treatment, |
| | field | clusters, sparing host tissues. | with resistant bacterial |
| | | | pockets. |
| Electrode | Surface electrodes | Directly targets bacteria on lead | Less effective against |
| Positioning | on lead | surface, disrupts early-stage biofilm. | mature biofilms. |
| | Encapsulated | Allows deeper field penetration, | More complex design; |
| | electrode design | targeting thicker biofilms. | potential interference with |
| | | | pacemaker function. |
| Electrochemical | Cathodic | Can generate hydrogen peroxide | Excessive ROS may |
| Effects | Stimulation (−) | (H₂O₂), which has antimicrobial | cause tissue damage. |
| | | effects. | |
| | Anodic | May lead to oxidative bacterial | Prolonged exposure |
| | Stimulation (+) | stress, disrupting cell function. | can corrode lead surfaces. |
| Treatment Duration | Short bursts | May impair biofilm development and | Might be insufficient for |
| (s - min - hrs) | (seconds) | bacterial adhesion. | established infections. |
| | Moderate | Greater biofilm weakening, bacterial | Risk of localized |
| | (minutes) | stress response. | pacemaker lead heating. |
| | Prolonged (hours) | Could fully eradicate mature | Host tissue exposure |
| | | biofilms, potentially making devices | concerns, pacemaker |
| | | self-cleaning. | function disruption risk. |

In one example, a device may include a lead or port lined with electrodes configured to deliver pulsed field energy for reducing or preventing microbial contamination along the implanted system. Instead of relying on numerous individually addressed electrodes, the design may employ two or more elongated conductors arranged in such a way that applying a controlled current and voltage results in a distributed electric field along the length of the lead. This configuration may simplify the electrical architecture and reduce the complexity of electrode management. By carefully selecting parameters such as field strength, pulse duration, and placement of the conductors, it may be possible to achieve effective bacterial disruption without causing undue harm to surrounding tissues, thus supporting long-term infection control.

In another example, the device may be configured to position at least one lead in the superior vena cava (SVC) or inferior vena cava (IVC). A self-expanding anchor could hold a lead securely in the SVC, where it could sense and stimulate the atrium while simultaneously gauging venous dimensions as a surrogate for volume status. If this lead setup achieves favorable electrical mapping and can narrow the QRS duration by selectively capturing conduction pathways, it might obviate the need for a left ventricular lead altogether, simplifying the system. The SVC may offer a stable anatomical reference point, allowing the device to measure subtle changes in venous dimension as an indicator of volume status. This measurement capability may be combined with atrial pacing functionality, enabling the lead in the SVC to both monitor hemodynamic parameters and support cardiac rhythm management. By optimizing the placement and configuration of the SVC lead, the device may achieve synchronized atrioventricular conduction, potentially resulting in a narrow QRS complex. Such optimization may reduce or eliminate the need for a separate left ventricular lead, thereby simplifying the overall system architecture. Alternatively, or in addition, the device may deploy any one or more of these concepts in the IVC.

In one example, the implantable device may be designed as a permanent platform intended to remain in place indefinitely. To support this long-term functionality, the system may incorporate features that reduce or negate the need for surgical explantation. For instance, the device may integrate pulsed field ablation or electroporation capabilities to manage infections as they arise. If an infection is detected, the system may initiate a sterilization protocol by applying the designated pulses along the lead, minimizing the likelihood of biofilm establishment or bacterial migration. Additionally, the device may store excess lead length within a circular housing, allowing a physician to rotate the unit to adjust the lead slack after implantation. This feature may simplify the implantation procedure and subsequent device management over time.

The device may further include one or more internal reservoirs configured to deliver therapeutic agents such as diuretics. Another reservoir may be included to hold air or a calibration fluid. Both reservoirs may be accessible through a refill port located in a convenient position, such as near the shoulder, enabling clinicians to replenish or adjust the device's fluid contents without resorting to invasive surgical interventions. With these integrated functionalities—pacing, sensing, infection control, and fluid management—the device may serve as a comprehensive, long-term solution for patients who would otherwise require multiple procedures or separate devices.

Parameters such as electrode placement, field delivery methods, reservoir configurations, and implantation sites may be refined through computational modeling, bench testing, and in vivo validation. By continuously optimizing these parameters, the system may offer reliable, multi-functional support that adapts to the patient's evolving clinical needs while maintaining a stable, long-lasting implantable platform.

In some examples, the system may incorporate a sheath to facilitate the placement, positioning, or temporary introduction of an electroporation apparatus. The sheath may serve as a structural guide for inserting leads, catheters, or other medical components into a desired location while maintaining stability during deployment. In one example, a sheath may be used to assist in the placement of pacing or defibrillator leads, ensuring proper alignment along cardiac tissue. This sheath may include a steerable or shape-memory element to direct the leads toward optimal anatomical regions, improving electrical capture and reducing unnecessary energy expenditure.

In some examples, a folding or shape-changing element may be incorporated into a lead deployment system to enhance positioning against the septal wall or other cardiac structures. This element may allow the leads to be dynamically positioned, expanded, or anchored in a manner that optimizes electrical capture, improving pacing efficiency and reducing energy consumption. The shape-changing element may comprise biocompatible, flexible, or memory-retaining materials, including shape-memory alloys, polymeric composites, or nitinol-based frameworks, that can transition between a delivery configuration and a deployed configuration in response to mechanical, electrical, or thermal activation.

In one example, the leads may be pre-embedded within a foldable framework that remains in a compact state during catheter-based delivery. Upon reaching the target site, the framework may unfold, expand, or fan out, conforming to the septal wall or other anatomical structures. This deployment mechanism ensures consistent tissue contact and electrical coupling with the myocardium, reducing the risk of lead migration or suboptimal pacing thresholds.

In another example, the shape-changing element may incorporate multiple lead contact points arranged in a grid, mesh, or segmented pattern. These leads may be selectively activated to map and adjust to the optimal conduction pathway, ensuring enhanced pacing efficiency. For example, a processor within the implantable device may assess electrical impedance or capture thresholds at various contact points and dynamically adjust which leads are activated to optimize conduction.

In some configurations, the folding or expanding structure may integrate a flexible electrode array, allowing for multi-site pacing, defibrillation, or conduction system targeting. This array may be capable of fine-tuned spatial adjustments, either passively via material properties or actively via an electromechanical actuation system. In one variation, the expanding framework may be self-anchoring, incorporating small atraumatic barbs, suction mechanisms, or compressive force against the cardiac tissue to maintain stable positioning without sutures or fixation screws.

In a further example, the shape-changing element may be used in conjunction with electroporation-based therapies, wherein leads embedded within the structure deliver pulsed electric fields (PEFs) to modulate myocardial conduction properties, ablate arrhythmogenic tissue, or perform targeted bacterial disruption near implanted cardiac devices. The combination of shape-adaptive lead placement with electroporation capabilities may provide a multifunctional platform for both electrophysiological therapy and infection control.

Additionally, in another example, the shape-changing lead structure may be delivered via a retractable sheath, allowing for precise placement within the heart before deployment. The sheath may guide the device through vascular pathways, and upon reaching the septal wall, may retract to allow the lead system to expand into its final anchored configuration. The sheath may also serve as a mapping or testing interface, ensuring that the placement achieves optimal conduction before final fixation.

In another example, a removable sheath may be utilized for the temporary introduction of an electroporation apparatus. This sheath may allow the electroporation system to be inserted, activated, and subsequently withdrawn once bacterial eradication or other treatment objectives have been met. Such a configuration may be particularly useful in applications where permanent electroporation electrodes are not necessary, such as periodic infection control in indwelling medical catheters, implantable reservoirs, or vascular access sites.

In some configurations, the sheath may include a folding, expanding, or shape-changing mechanism to facilitate the deployment of multiple leads or electrodes. For example, in a pacemaker or ICD system, the sheath may be designed to deliver multiple leads onto the septal wall of the heart. The sheath may incorporate a flexible or expanding structure that unfolds upon reaching the implantation site, allowing leads to be spread across a larger surface area for optimal ventricular capture and conduction system engagement. This approach may improve pacing efficiency and reduce the risk of lead dislodgment by distributing electrical stimulation across a broader region of the myocardium.

Additionally, in another example, the sheath may be designed with embedded electrodes to provide an initial mapping or testing function before permanent lead placement. These electrodes may deliver a low-energy test pulse to assess local tissue responsiveness, enabling real-time optimization of lead placement prior to fixation. Once the optimal location is determined, the sheath may retract or dissolve, leaving only the implanted leads behind.

In yet another example, a sheath-integrated electroporation assembly may be configured to sterilize a vascular entry site before catheter insertion. This sheath may include circumferential electrodes that deliver short, high-voltage pulses to disrupt bacterial biofilms before advancing a catheter, reducing the risk of bloodstream infections. The sheath may then be withdrawn, leaving the primary catheter or medical device in place.

These sheath-based configurations may be used in a variety of electroporation-enabled medical applications, including cardiac pacing, infection control, and vascular access management. By incorporating expandable, steerable, or retractable elements, the sheath may enhance procedural precision, minimize complications, and enable adaptive deployment of electroporation-based treatments in both temporary and permanent medical interventions.

In some examples, an electroporation or pulsed field ablation (PFA) device may be configured as a deployable structure designed to conform around a target medical device, tissue, or anatomical site. This deployable electroporation assembly may be removable, semi-permanent, or in some cases, permanently positioned, providing flexible treatment options for infection control, biofilm disruption, tissue modulation, or conduction pathway modification.

In one example, the electroporation device may be delivered via a catheter or sheath and expand upon deployment, forming a conformal structure around a catheter, implanted lead, cardiac device, stent, vascular graft, or other medical implant. The deployable electrodes may be composed of flexible conductive materials, such as metallic meshes, segmented electrode arrays, or conductive polymer coatings, allowing them to wrap, adhere, or contour to the shape of the target site. The electrodes may be configured to generate targeted electric fields, applying controlled electroporation pulses to disrupt bacterial membranes, prevent biofilm formation, or modulate cellular function in the surrounding tissue.

In another example, the deployable electroporation device may be designed for temporary use, allowing it to be positioned, activated, and subsequently removed after treatment is complete. This may be particularly beneficial in post-surgical sterilization procedures, catheter decontamination, or transient infection management, where continuous electroporation is not required. In some variations, the removable device may feature a self-expanding scaffold that automatically deploys upon insertion and collapses for retrieval via a retraction mechanism, sheath, or guidewire-assisted withdrawal.

In a further example, the electroporation device may be semi-permanent, remaining in place for prolonged treatment durations but allowing for removal or replacement if needed. This may be advantageous in scenarios where periodic bacterial control or long-term infection prevention is necessary, such as within peripherally inserted central catheters (PICCs), dialysis access devices, or cardiac implants. The semi-permanent system may include bioresorbable components, gradually degrading over time to eliminate the need for a retrieval procedure while still ensuring prolonged therapeutic efficacy.

In some configurations, the electroporation/PFA device may be permanently affixed to a target medical implant, allowing for continuous or on-demand pulsed field therapy to maintain sterility and device function over extended periods. For example, a pacemaker, implantable cardioverter-defibrillator (ICD), or vascular stent may integrate a deployable electrode array that remains conformed to the device's outer surface, delivering electroporation therapy as needed to prevent biofilm formation, optimize tissue integration, or facilitate targeted ablation of arrhythmogenic tissue. In some examples, the permanent system may include a wireless power transfer mechanism or an integrated battery, allowing for programmable or clinician-triggered electroporation cycles without requiring direct wired connections.

In additional examples, the deployable electroporation device may include adaptive or shape-memory materials, such as nitinol, biocompatible conductive polymers, or hydrogel-based structures, that can self-adjust to conform to irregular surfaces, ensuring uniform electroporation coverage. The electrodes may be arranged in a segmented, ring-based, or matrix configuration, allowing for precise field shaping and spatially targeted energy delivery based on the clinical indication.

These deployable electroporation systems may be configured for use in a variety of medical applications, including infection prevention in implantable devices, cardiac tissue modulation, vascular graft sterilization, and catheter-associated infection control. By integrating removable, semi-permanent, and permanent electroporation configurations, the system enables customized therapeutic approaches, enhancing device longevity, improving patient outcomes, and reducing the need for antibiotic-based infection control strategies.

In another example, incorporating pulsed field ablation (PFA) capabilities into a pacemaker-like system designed for permanent implantation could reduce device explants due to infection. If the system senses an early stage of bacterial colonization or receives a physician-triggered command, it could deliver short, high-intensity pulses to sterilize the surrounding tissue and device surfaces. This could extend the functional lifespan of the device and reduce the need for risky revision surgeries, which is particularly attractive given that pacemaker and ICD leads are often placed in anatomically challenging locations.

Finally, the notion of a compact, integrated device that manages all these functions—pacing, sensing, infection control, and fluid management—is compelling. A circular hub that stores extra lead length, adjustable by simply rotating the device to achieve the right amount of slack, could streamline implantation and adjustment procedures. Incorporating at least two reservoirs—one for introducing air or another inert gas for calibrations or pressure measurements, and one for delivering diuretics—would further enhance the device's capabilities. Physicians could refill these reservoirs through a convenient shoulder-accessible port, ensuring long-term functionality. With careful engineering, materials selection, and iterative design, such a multi-purpose, permanently implantable system could represent a significant advancement in patient care, reducing complications, and enhancing treatment efficacy for a wide range of conditions.

In one example, a standalone implantable reservoir device may be configured for long-term implantation in various anatomical locations, such as within a left pectoral region (similar to a pacemaker pocket) or in the abdominal cavity. This device may include a refillable port accessible through the patient's skin, allowing healthcare providers to introduce a range of therapeutic agents. For heart failure management, diuretics may be stored within the reservoir, slowly diffusing into the surrounding tissue or fluid spaces. Such diffusion could occur even without direct intravascular access, as the medication may be absorbed through local tissues, eventually achieving systemic levels.

In one example, the reservoir device may communicate wirelessly with other implantable systems, such as a left atrial appendage (LAA) device, a pacemaker (PM), or another implanted sensor or controller. These other devices may deliver subthreshold pulses or low-level electrical signals to the reservoir device, effectively serving as a communication channel. Through this communication protocol, the reservoir device may receive instructions to adjust dosage, initiate a treatment cycle, or activate additional functionalities.

In one example, the reservoir device may incorporate a pulsed field ablation (PFA) capability to maintain a sterile environment around its surfaces. The internal surfaces of the reservoir and the associated diffusion membranes could be lined with electrodes. When triggered, these electrodes may apply short, high-intensity electric fields to disrupt bacterial cell membranes, inhibiting the formation of biofilms. By doing so, the device may mitigate or eliminate infections without the need for external interventions. Furthermore, if the reservoir is filled with antibiotics or other antimicrobial agents, the combination of chemical and electrical strategies may provide a robust, multifaceted approach to clearing infections around the device.

In another example, the reservoir device may be designed to release medication in a 360-degree pattern, providing even distribution in the local tissue environment. If contamination is detected—whether through direct sensing within the device or by receiving communication signals from other implanted devices that have detected changes in patient status—the reservoir device may deliver an antibiotic load while simultaneously initiating PFA cycles. This dual-pronged approach may substantially reduce the likelihood of persistent or recurring infections.

In one example, the device's power needs for PFA could be met through a physical connection port, inductive coupling, or by receiving low-level electrical energy transferred from another implanted system. Over time, as clinicians refine dosing strategies, communication protocols, and sterilization parameters through computational modeling and in vivo testing, the reservoir device may evolve into a versatile, general-purpose platform. It could be applied not only in heart failure management but also as an adjunct to pacemakers, implanted joint prostheses, spinal devices, or chemotherapy ports, ensuring a high standard of infection control while maintaining continuous, localized therapeutic delivery.

In one example, a permanently implanted device may include features that allow the application of pulsed field ablation (PFA) or electroporation to sterilize implanted leads, ports, or other components. Such a device could incorporate electrodes or electrically conductive wires arranged along the surface of a lead. When activated, these conductors may deliver high-voltage pulses that generate intense electric fields, disrupting bacterial membranes and preventing biofilm formation. This functionality may help maintain device sterility over time, reducing the need for repeated surgical interventions.

In another example, the device may require a dedicated power source or connection interface to deliver the required PFA pulses. A direct physical connection, such as a needle-like plug or a dedicated implantable port, could allow a clinician to couple an external energy source to the implanted system. Through this interface, short, high-intensity pulses may be applied on-demand, for instance, when routine monitoring suggests early signs of bacterial contamination. Alternatively, the connection may be established through inductive coupling or other wireless energy transfer methods, though direct conductive access may provide more reliable and controlled delivery of the pulsed fields. Such a system could be integrated into devices originally intended for chemotherapy delivery or pain management in spine or joint applications, enabling sterilization protocols if these regions become infected.

In one example, this sterilization strategy may be combined with an implantable drug reservoir system designed to deliver medications such as diuretics. A multi-chamber reservoir, which may include one or more balloons or flexible pouches, could be placed in a location commonly used for cardiac devices, such as the left shoulder region. This region may host an implanted pacemaker or defibrillator system. In some configurations, the diuretic reservoir may be positioned contralaterally—e.g., in the right shoulder—or even in the abdomen if larger volumes are necessary. This arrangement could facilitate easy access for periodic refilling of the diuretic or other medications by inserting a needle through the skin into a dedicated port. Such refill procedures may occur weekly, monthly, or on another schedule as determined by the patient's clinical needs.

In one example, the sterilization capabilities afforded by the PFA system may also be applied to these drug reservoir access points. By lining the internal surfaces of the refill port or associated conduits with electrodes, it may be possible to initiate a brief PFA cycle following a refill procedure to reduce the risk of bacterial ingress and subsequent infection. This could help maintain the long-term integrity of both the drug delivery and electrophysiological functions of the device. For instance, after refilling the diuretic reservoir at the shoulder site, the system could deliver a short series of pulses to sterilize the needle track and port interface, reducing bacterial contamination risk without requiring separate sterilization interventions.

In one example, the integrated system may combine PFA-based sterilization, drug delivery, and cardiac support into a comprehensive platform. The device may include sensors to monitor physiological parameters, such as fluid status or cardiac rhythm, and adjust therapy accordingly. By incorporating diuretic delivery capability alongside a pacemaker or ICD, the system may respond to changes in patient fluid balance, administering diuretics as needed to manage volume overload. If the device senses early signs of infection, such as subtle changes in impedance or other indicators, it may apply a sterilization protocol through the PFA module to inhibit bacterial growth at the device interface. Over time, iterative refinement of these parameters, informed by computational modeling, bench testing, and in vivo studies, may optimize the balance between effective bacterial disruption and patient safety.

In one example, the device's leads may be designed with slack stored in a circular housing. Rotating the device could adjust lead length without additional surgery. This mechanical flexibility may simplify initial implantation and long-term maintenance. By integrating diuretic reservoirs, PFA capabilities, and pacing or defibrillation functions into a single platform with easily accessible refill and sterilization mechanisms, the device could reduce the frequency of invasive procedures, lower infection risk, and improve overall patient management.

In one example, incorporating advanced sensing capabilities may further enhance the overall function of the device. For instance, integrating chemical or biosensors capable of detecting early markers of infection or shifts in local tissue chemistry could enable more proactive interventions. Upon identifying bacterial growth or biofilm formation, the device may initiate an automated PFA cycle, release a therapeutic agent, or send a wireless alert to an external reader, prompting early intervention and reducing the risk of clinical complications.

In another example, the device could benefit from enhanced communication protocols. While subthreshold pacing pulses from another implant may serve as a basic communication link, more sophisticated wireless transmission (such as low-frequency RF or ultrasound-based signaling) could allow for more robust data exchange. This might include adjusting therapy parameters, updating firmware, or synchronizing multiple implanted devices for coordinated, system-wide responses—such as increasing diuretic delivery when heart failure sensors detect fluid overload, or intensifying sterilization when microbial signatures are detected.

In one example, the mechanical design of the device may also be optimized for long-term stability. For devices placed in dynamic anatomical sites—such as near joints or in regions subject to muscle movement—flexible materials or shape-memory alloys could be incorporated to maintain a secure yet adaptable fit over time. Additionally, advanced coatings or surface treatments, such as antimicrobial peptides or nanostructured surfaces, may complement PFA-driven sterilization by reducing initial bacterial adhesion and colonization.

In another example, methods of energy delivery and storage might be considered. While direct needle connections or inductive coupling are mentioned, exploring alternative approaches for sustaining device functions could be beneficial. For instance, including an energy-harvesting component—such as piezoelectric materials that convert mechanical motion into electrical energy—may reduce the frequency of external charging or power-ups. This may facilitate a more autonomous, maintenance-free device capable of functioning for extended periods without patient inconvenience.

In one example, integration of targeted drug release strategies could be refined. Although continuous diffusion of medication from the reservoir into surrounding tissues may be sufficient for some applications, adjustable release rates or pulsatile delivery patterns may better match clinical needs. For instance, the reservoir could be configured to deliver a diuretic at higher concentrations during certain times of day or in response to sensor-detected increases in venous pressure, thereby tailoring therapy to the patient's dynamic condition.

Additionally, considering long-term tissue compatibility and overall biocompatibility could lead to the incorporation of biodegradable components, removable or replaceable reservoirs, or modular attachments that can be exchanged through minimally invasive procedures. For example, if a particular segment of the device's lead or reservoir becomes heavily colonized or damaged, a modular design might allow for partial replacement rather than full explantation.

In some examples, the device may be integrated with or connected to pacing or defibrillation leads. By doing so, the device may function as a comprehensive therapeutic platform, managing both pharmacological treatments and cardiac rhythm. In one example, if the patient exhibits fluid overload signals, the device may increase diuretic dosing while simultaneously adjusting pacing parameters to optimize hemodynamics. Should the patient develop an arrhythmia, the device may deliver antiarrhythmic drugs and provide pacing or defibrillation therapy.

In another example, sensors integrated into the cardiac leads may provide real-time ECG data, enabling the processor to adapt medication regimens based on current cardiac function. If the device detects signs of infection around the lead entry points, it may initiate an antibiotic dose and follow up with electroporation near these leads to reduce bacterial colonization.

In one example, the device may include a variety of sensors to guide therapy. For example, pressure sensors may measure venous pressure, aiding in early detection of fluid overload. Biochemical sensors may monitor drug concentrations, blood biomarkers of infection (e.g., inflammatory markers), or electrolyte balance. Cardiac leads may serve double-duty as

Closed-loop control may allow the device to dynamically adjust dosing regimens. In one example, if fluid overload is detected via pressure or impedance sensors, the processor may increase the delivery of a diuretic reservoir. If markers of infection arise, the device may adjust antibiotic dosing and trigger an electroporation cycle. This adaptive approach may reduce the burden on patients and providers, offering a more consistent and responsive therapy paradigm.

While electroporation or PFA may serve as the primary active infection control measure, the device may also incorporate additional strategies. For example, the interior surfaces of the reservoirs, pumps, and fluid lines may be coated with antimicrobial materials such as silver particles or antibiotic-impregnated polymers. In one example, the device may include a small secondary reservoir containing an antimicrobial flushing solution. After each refill, this solution may be dispensed to rinse the fluid channels, followed by an electroporation cycle.

In another example, the device may employ ultraviolet (UV) or blue-light LEDs aimed at internal fluid pathways. Brief light exposure may help disrupt bacterial growth. Such optical sterilization methods may be used in conjunction with electroporation for a multifaceted infection prevention approach.

In some examples, the device may be placed subcutaneously in the chest region, particularly suitable for patients also requiring cardiac leads. However, the device may alternatively be implanted in the abdomen, pelvis, or even placed intraperitoneally, depending on the therapeutic goals. For patients requiring targeted drug delivery to abdominal organs, abdominal implantation may simplify direct access to relevant blood vessels or tissue compartments.

In one example, larger reservoir volumes may be achieved by placing the device in the abdominal cavity. Such examples may benefit patients requiring continuous chemotherapy or long-term antibiotic infusion. Different anatomical sites may also allow for the integration of different sensor arrays, optimizing therapy personalization.

In one example, the device may include a rechargeable battery system. External inductive chargers or energy harvesting methods (e.g., from patient motion or thermal gradients) may extend battery life. The device may also incorporate wireless communication capabilities, allowing clinicians to remotely adjust dosing, query sensor data, and update electroporation parameters. Encrypted data transmission may ensure privacy and security.

In one example, the device may include two reservoirs: one for a loop diuretic to manage fluid overload and another for a broad-spectrum antibiotic. The device may integrate pacing leads to monitor and manage arrhythmias. If fluid sensors detect increased venous pressure, the diuretic delivery may increase, while the pacing rate may be adjusted to improve cardiac output. If infection indicators rise, an antibiotic bolus may be delivered and the electroporation assembly activated to sterilize the refill port.

In another example, the device may be implanted in the abdomen and hold chemotherapy agents. Periodic refills may be performed by the oncologist, and each refill may be followed by an electroporation cycle to prevent bacterial contamination. Integrated sensors may monitor tumor markers and adjust dosing accordingly.

In yet another example, the device may incorporate a three-reservoir system-one containing a diuretic, one an antibiotic, and one a cardiovascular agent. The device may combine electroporation electrodes and a UV LED sterilization unit near the port to provide layered anti-infection defenses. Cardiac leads and sensors may be included for comprehensive patient management.

The components of the device may be manufactured using known biocompatible materials and techniques to ensure long-term stability and patient safety. All interfaces between reservoirs, ports, and internal electronics may be hermetically sealed. The reservoirs, membranes, pumps, and leads may be validated through fatigue testing, sterilization validation, and compatibility studies with the intended drug formulations. Regulatory approvals may be pursued according to applicable standards to ensure the device's safety and efficacy in human patients.

In one example, the reservoir may take a low-profile, disk-like shape, with a flattened, rounded geometry designed to fit comfortably in subcutaneous or submuscular pockets. Such a shape may minimize patient discomfort, reduce pressure points against underlying tissues, and permit secure anchoring under the skin. In another example, a more elongated, cylindrical reservoir could be utilized, offering streamlined placement in narrower anatomical pathways, such as along the chest wall or in the abdominal region.

In one example, the reservoir may incorporate a flexible, pouch-like design constructed from a layered polymeric membrane. For instance, an inner lining of medical-grade silicone or polyurethane could provide biocompatibility and maintain drug stability, while an outer layer of expanded polytetrafluoroethylene (ePTFE) or porous polyethylene could enhance tissue integration and minimize fibrous encapsulation. Alternately, incorporating a stiff polymer or titanium-based framework could provide a more rigid structure, ensuring dimensional stability and preventing deformation over time.

In another example, the reservoir may be composed of segmented chambers or multiple compartments, each separated by a thin membrane or valve system. Such segmentation may allow for the storage of multiple drugs or different concentrations of a single agent. A modular approach, where each segment can be independently refilled or replaced, could facilitate dynamic therapy regimens without requiring multiple reservoir implants. This configuration could be achieved by nesting smaller polymeric bladders within a larger rigid housing, or by employing internal baffles that maintain shape and prevent mixing of contents.

In one example, incorporating a spring or shape-memory alloy framework into the reservoir wall may allow it to expand or contract as drug volume changes, maintaining consistent internal pressure. This could help ensure stable dosing and predictable release kinetics. A collapsible design might involve a bellows-like structure made from biocompatible metals or polymers, permitting controlled compression and expansion without compromising reservoir integrity.

In another example, an outer surface coating could be applied to promote favorable tissue integration, reduce infection risk, or improve device palpability for clinicians. For instance, a hydrophilic coating may encourage fibrous ingrowth that stabilizes the device's position, while antimicrobial coatings could reduce the colonization risk. A textured or patterned external surface could provide frictional hold, reducing the likelihood of device migration over time.

In one example, the reservoir may be designed with integrated channels or microfluidic passages that facilitate controlled diffusion of the stored medication into surrounding tissues. Such microfluidic features could be etched into a rigid polymer or metal shell, or embedded within a flexible silicone membrane. By adjusting channel dimensions, it may be possible to fine-tune release rates. Additionally, incorporating a selectively permeable membrane—such as one with nanopores—could allow passive diffusion of certain molecules while retaining others.

In another example, the reservoir may be engineered for easy percutaneous access. A self-sealing silicone port placed at a shallow depth could enable clinicians to refill the reservoir without complex surgical intervention. The shape around the port region may be slightly raised or include tactile markers to help guide needle insertion. A funnel-like internal geometry or specially contoured surfaces could direct the needle tip into the designated chamber, preventing accidental punctures of the reservoir walls.

In one example, structural integration with other implanted components may influence reservoir shape and material selection. If the reservoir is intended to connect with leads, sensors, or other device modules, a branched or multi-lobed design might accommodate these attachments. Rigid flanges or snap-fit connectors molded into the reservoir housing could interface with a pacemaker's anchoring bracket or a separate drug delivery catheter, ensuring a stable mechanical assembly.

In another example, the reservoir might incorporate radiopaque markers or embedded RFID tags to facilitate imaging and identification. Material selection, such as incorporating tantalum markers or barium sulfate-loaded polymers, may help practitioners locate the reservoir precisely during follow-up procedures. Such markers could be distributed in a pattern around the device's circumference, allowing orientation to be readily determined.

In one example, if the reservoir is intended for high-volume applications, a larger, dome-shaped design made from a flexible, high-tensile-strength polymer might be chosen to accommodate expansions in drug volume. For lower-volume, precise dosing scenarios, a smaller, more rigid reservoir with a limited expansion range might be preferred to maintain strict control over drug egress.

In another example, long-term durability and fatigue resistance may guide material choice. High-performance polymers like polyether ether ketone (PEEK) or titanium shells could be considered for their structural integrity and corrosion resistance. For drug-contacting surfaces, inert elastomers or fluoropolymers might ensure that stored medications remain stable over extended periods.

In one example, consider the refill port assembly. This port may be designed as a small, raised structure on the device's housing, featuring a self-sealing elastomeric membrane that allows repeated needle insertions without significant wear. The port could include angled internal guides or tapered channels that help direct the refill needle into the intended reservoir chamber, reducing the risk of damage to the reservoir walls. To improve user confidence during refills, the outer surface of the port may incorporate a tactile pattern, radiopaque markers for imaging guidance, or a slightly thicker rim that is easily palpable through the skin.

In another example, leads and connector systems may be developed to integrate seamlessly with the reservoir device. Leads running from a processor or power source to the reservoir's internal electrodes might be routed through dedicated cable channels or feedthroughs within a rigid housing. To ensure long-term reliability and minimize mechanical stress, these leads may be coiled or zigzagged internally, providing slack and flexibility that help absorb motion over the patient's lifetime. The connectors that link these leads to external modules—such as a temporary external power supply for PFA—could be designed as keyed, hermetically sealed ports, ensuring a secure, correct fit every time.

In one example, if the system incorporates electrodes for pulsed field ablation (PFA) or infection control, these electrodes could be integrated along the inner or outer surfaces of the reservoir housing. They might be printed as thin metallic layers on a polymer substrate or formed as discrete, embedded wires arranged in a grid or circumferential pattern. Insulating layers may separate electrodes from direct tissue contact except where ablative energy is intended. By selecting flexible yet durable conductive materials—such as platinum-iridium alloys or conductive polymers—electrode arrays can maintain consistent performance despite anatomical motion or long-term implantation.

In another example, a mechanical anchor or fixation system could be integral to the device's design. For instance, small fins, hooks, or wings might be molded into the device's outer shell to help secure it in a stable position within a tissue pocket. In the case of devices placed in or near the vasculature, a self-expanding frame made of nitinol could gently press against vessel walls, anchoring the device without sutures. Alternatively, for subcutaneous placement, textured surfaces or microporous coatings may encourage tissue ingrowth to stabilize the device over time.

In one example, if the device requires a dedicated fluid line for either infusion or withdrawal of fluid, the tubing and associated connectors could be constructed of kink-resistant materials such as reinforced silicone or braided polyurethane. Strain-relief features might be added where tubes connect to the reservoir, preventing stress concentrations that could lead to cracks or leaks. Quick-disconnect connectors with one-way valves might permit safe and easy attachment of external pumps or syringes when adjustments are needed.

In another example, if a battery or energy-harvesting unit is integrated, the mechanical design could include a dedicated compartment or cavity within the device's housing. This compartment might be lined with vibration-damping materials or flexible mounting brackets to protect sensitive electronics from mechanical shocks. Additionally, incorporating a hermetic sealing method—such as laser welding for metal housings or advanced bonding techniques for polymers—would maintain internal dryness and protect electronic components from bodily fluids. Vents or pressure-equalizing membranes could be included to accommodate slight internal pressure changes without compromising the seal.

In one example, integration with sensing elements—such as pressure transducers, flow sensors, or chemical detectors—may influence mechanical design choices. Sensors could be embedded directly into the reservoir wall or positioned along fluid channels. The housing could incorporate transparent or translucent regions (using biocompatible polymers) to facilitate optical sensors, or recesses and flat surfaces for stable sensor mounting. A thin partition of a specific polymer could serve as an acoustic window for ultrasound-based sensors, ensuring reliable signal transmission.

In another example, modularity and upgradability might be considered. The device could be designed in separable sections: one containing the reservoir and electrodes, another housing electronics and power components. Interconnecting sections with sealed, mechanical couplings might enable selective replacement or upgrading of certain device portions without removing the entire assembly. Such couplings would need to ensure long-term mechanical stability, low-profile geometry, and enduring biocompatibility.

In one example, the external contouring of the device might be shaped to minimize shear forces against surrounding tissues. Smooth transitions, rounded edges, and low-profile protrusions can lower the risk of patient discomfort and reduce the potential for erosion through the skin. Strategically placed grooves or channels on the device surface might encourage vascular or fibrous ingrowth patterns that stabilize the device while avoiding stress points that could lead to tissue irritation.

In another example, if the device is intended for use in dynamic anatomical environments—such as near joints or musculature that undergoes frequent movement—selecting flexible materials or hybrid rigid-flex designs could ensure durability. For instance, incorporating a flexible polymer “hinge” region between a rigid reservoir section and a rigid electronics section could help the device accommodate motion without placing undue stress on leads or fluid lines.

In one example, the method by which the device interfaces with external energy sources or programming units could be explored further. If the system is designed to receive power or instructions for PFA treatments, consider how a stable and repeatable mechanical connection might be achieved. This could involve a specialized mating surface or dock that aligns precisely with an external applicator. Such alignment features might include tapered insertion guides, latch mechanisms, or magnetic alignment points that draw the external connector into the proper position without excessive manual manipulation. A robust sealing surface—perhaps using a compressible O-ring or a gel-like interface layer—could maintain a fluid-tight barrier while the external unit engages with the implanted device.

In another example, mechanical considerations related to modular components warrant further attention. If the device is intended to accept upgradeable or replaceable modules—such as an enhanced sensor package, a larger reservoir segment, or an updated electrode array—a reliable mechanical coupling strategy must be considered. Precision-engineered interlocks, snap-fit interfaces, or keyed, rotational couplings could ensure that replacement modules seat correctly and maintain proper alignment of fluid paths, electrical connectors, and mechanical support structures. This may allow for incremental device improvements over time without necessitating full system removal.

In one example, the mechanical layout of integrated sensors could be refined. Certain sensor modalities, like optical or ultrasound-based units, may require clear line-of-sight or acoustic windows. Accommodating these needs might involve shaping transparent or acoustically transmissive inserts in the device housing. The mechanical integrity of these inserts, their bonding method to the main housing material, and their ability to maintain properties under physiological conditions could all influence long-term sensor function. Additionally, ensuring that sensors remain stable under repetitive mechanical stresses, such as muscle contractions or patient movements, might involve internal shock-absorbing features or damping layers.

In another example, considering the mechanical aspects of drug diffusion membranes or nanoporous channels may offer improvements in dosing precision. Fine-tuning channel geometry, wall thickness, and spacing could allow for more predictable release kinetics. Integrating microvalves or collapsible, spring-loaded shutters might enable controlled pulses of medication rather than a continuous, passive diffusion. The mechanical reliability of these miniature features under years of implantation and the case with which clinicians could adjust them via external programming should be evaluated.

In one example, it might be valuable to examine how the device's mechanical design could facilitate manufacturing, sterilization, and regulatory compliance. Choosing materials that can withstand common sterilization procedures—such as ethylene oxide gas, gamma irradiation, or autoclaving—without deforming or degrading may influence the final production workflow. Features like integrated positioning jigs, assembly guides, or indexing marks within internal chambers might simplify manufacturing steps and quality control inspections.

In another example, ergonomic considerations for clinicians who interact with the device could be incorporated. During refill procedures or when connecting to external units, having thumb rests, tactile ridges, or contoured surfaces on the device's outermost layer (as perceived through the patient's skin) might help physicians locate the device and port with minimal reliance on imaging. Such subtle mechanical cues could streamline procedures, reduce the chance of needle misalignment, and ultimately improve the patient's experience.

In one example, the design of interior cavities used for fluid or air storage could be further elaborated. For instance, if the system stores both a drug solution and a calibration fluid or air pocket, the internal partitioning could be made from semi-rigid polymers that resist mixing of the two media. Valve systems or slidable partitions could segregate these compartments until external manipulation—such as a specific pattern of pressurization or vacuum application—triggers mixing or displacement. The mechanical integrity of these partitions, as well as their endurance under cyclical loading from refills and drug withdrawal, may dictate long-term reliability.

In another example, surface texturing or patterning could be considered to optimize fluid flow or encourage specific tissue responses. Micro-grooves or ridges on the internal surfaces of fluid channels might reduce turbulence, enhance laminar flow, and promote stable drug distribution. On external surfaces, certain textures may encourage a controlled fibrous encapsulation that secures the device over time, or conversely, reduce adhesion to minimize tissue ingrowth if long-term removability remains a consideration.

In one example, the device's electrical design may include a low-power microcontroller or processor embedded within the implant, responsible for managing routine operations, such as monitoring reservoir levels and sensor data. The processor could remain in a low-power state for most of the time, periodically waking to check fluid levels (via pressure or optical sensors), battery charge state, and any physiologic parameters—such as temperature or impedance changes indicative of infection risk. When fluid levels approach a preprogrammed threshold, the device could communicate wirelessly to an external reader or patient alert system, prompting the patient or physician to schedule a refill procedure.

In another example, the device may incorporate a memory unit and data logging capabilities. Over the course of a month (or another designated interval), the device could store information on drug release rates, reservoir volume trends, and sensor readings indicative of patient response or early infection signals. This data could be retrieved non-invasively during a follow-up appointment using a handheld programmer or a clinic-based console. By reviewing these logs, clinicians may adjust dosage parameters, refill schedules, or even preemptively plan a sterilization cycle if infection risk appears to be rising.

In one example, delivering the high-energy pulses required for electroporation or pulsed field ablation may exceed the power capacity of a small, permanently implanted battery. Instead, the device could be designed to receive power from an external source during scheduled office visits. For instance, an external handheld power unit might connect to the implant via a transcutaneous needle-based port or through an inductive coupling interface. Once connected, the external unit could deliver a controlled burst of high-voltage pulses to the reservoir's integrated electrode array, effectively sterilizing the device's surfaces. The electrical design would include protective circuitry—diodes, transient voltage suppressors, and carefully chosen capacitors—to safeguard the internal electronics from the high-voltage environment created during PFA.

In another example, the electrical design may incorporate a hybrid power system. A small, long-lasting battery or energy harvester (e.g., thermoelectric or piezoelectric elements) might power low-demand functions like reservoir monitoring, sensor polling, and data logging. For high-demand events like PFA, the device could feature an internal energy storage element, such as a capacitor bank, which charges slowly over time from the internal battery or from periodic external transmissions of energy. During an office visit, a physician could activate a mode on the external programmer that delivers a brief, high-current charge wirelessly to the implant. The device's capacitor bank would store this energy and release it in carefully timed pulses to achieve electroporation. This approach could reduce patient inconvenience and risk by avoiding frequent battery replacements or large internal power cells.

In one example, software algorithms running on the implant's microcontroller could implement closed-loop control strategies. For instance, if the device detects subtle changes in local impedance—potentially indicating bacterial growth—it might prompt the patient or physician with a notification and recommend scheduling a sterilization session. The same software could manage dosing schedules for the reservoir. By analyzing historical usage patterns, the device's software could refine the estimated depletion rate of the stored drug. When it calculates that the reservoir will reach a predetermined “low” level within a certain timeframe, it could send a message to a patient's home monitoring station, a smartphone-based companion app, or a clinician's portal, suggesting a refill appointment.

In another example, communications protocols for data and control instructions may rely on low-frequency RF or magnetic induction-based communication. To maintain patient safety and data security, the device's software could implement encryption and authentication measures. During a refill or maintenance visit, the physician could use a secured, handheld programmer that transmits encrypted commands to the implant, adjusting parameters like infusion rates, sensor polling intervals, and sterilization readiness. The device's internal software would verify the identity and authorization of the programmer before accepting any changes.

In one example, the external software environment—such as a clinic-based workstation—could maintain a comprehensive patient record, integrating data from the implant with other medical records. This external software might run predictive analytics, forecasting the patient's future medication needs, risk of infection, or potential cardiac arrhythmias if connected to a pacemaker module. Such predictive insights could guide the physician in fine-tuning the device's programming. The workstation software might also generate compliance reports, ensuring that refills and maintenance procedures occur on schedule.

In another example, fail-safe and redundancy measures may be integrated into the electrical and software design. If a sensor fails or a drug delivery valve malfunctions, the software could detect abnormal readings and initiate a fallback mode. For PFA sterilization, the software might run self-check routines before initiating high-voltage pulses, confirming that leads and electrodes are intact and that capacitors are charged to the correct levels. If any anomaly is detected, the device could abort the procedure and notify the clinician.

In one example, the walls of a peripheral intravenous catheter (PIV) or peripherally inserted central catheter (PICC) include embedded electrode strips spaced at regular intervals along the catheter's length. These strips can be activated briefly to deliver high-voltage pulses, disrupting bacterial cells that adhere to the catheter surface. A small connector or lead wire near the catheter hub allows the attending clinician to connect an external power source intermittently, applying electroporation pulses either on a preset schedule or as-needed when sensors detect elevated bacterial counts. Similarly, a Foley catheter may incorporate ring-shaped electrodes around its circumference, particularly near the insertion site in the bladder. These rings may be powered by a compact control unit integrated into the catheter's external port. The control unit can measure impedance in the urinary tract, trigger electroporation if bacterial biofilms are detected, and log usage data for subsequent review.

In another example, the systems may include a subcutaneous port commonly used for drug infusion (e.g., chemotherapy or long-term antibiotic therapy) but enhanced with small electrode arrays near the silicone septum. Whenever the port is accessed with a needle, electroporation pulses can be delivered to sterilize the immediate area, reducing the risk of bacterial colonization. In certain examples, an implantable drug reservoir includes multiple internal compartments, each possibly containing different therapeutic agents. Electrodes arranged around the fluid pathways or refill septum provide prophylactic or therapeutic electroporation. A control module regulates the timing of these pulses—for instance, automatically administering them immediately before or after each refill to minimize contamination risk. Yet another example contemplates orthopedic hardware (e.g., plates, rods, screws) that incorporate electrode surfaces on or near their anchoring points. A small embedded or attachable power interface can deliver pulsed fields to the implant surface to combat infection in cases of open fractures or high-risk surgical sites. In some examples, the femoral or tibial component of a knee prosthesis includes an integrated electrode grid at the interface with bone or soft tissue. A patient-specific power pack or external system can couple electromagnetically or via a transcutaneous port to provide short bursts of pulsed fields. This approach may hinder biofilm formation on the prosthetic surfaces, helping preserve implant integrity over time.

Sensors embedded along the catheter or implant surface may measure changes in electrical impedance correlating with bacterial adhesion. If an increase in impedance is noted—often indicative of biofilm formation—the control unit can initiate an electroporation cycle. In alternative examples, small optical or pH sensors reside within the catheter lumen or near the implant surface. These can detect shifts in pH or the presence of fluorescent markers associated with bacterial growth, signaling the system to apply pulsed fields. Some examples may incorporate single-use strips or integrated biochemical sensors that detect specific bacterial enzymes. Once triggered, the system can automatically deliver the electroporation pulses or log an alert for manual activation. The control unit may measure temperature at the treatment site to confirm that electroporation pulses do not excessively heat adjacent tissues. If temperatures rise above a set threshold, the software can shorten pulse lengths or decrease voltage to maintain safety. For catheters placed in dynamic fluid environments (bloodstream, bladder), flow and pressure sensors can confirm adequate fluid exchange and help differentiate infection-related blockages from mechanical occlusions.

Many examples anticipate that a clinician will connect a small power cable or specialized needle to the external hub, administering high-voltage pulses on an as-needed basis. This approach reduces the permanent implant footprint and extends device life by outsourcing the bulk of the power generation to an external system. In another example, a coil or magnetic coupling is integrated into the subcutaneous port or implant, allowing an external inductive power transmitter to deliver enough energy for short bursts of electroporation. This removes the need for transcutaneous leads but still relies on external hardware for major power demands. Some devices include a capacitor that charges slowly over time from a small battery or harvesting component (e.g., from body motion or temperature differentials). Once sufficiently charged, the capacitor can release a high-voltage pulse sequence. This can be useful for automated, periodic sterilization without external intervention. In examples requiring frequent treatments, a rechargeable battery within the implant can be re-energized via inductive charging pads placed on the skin. Built-in power management circuitry ensures that the system can deliver consistent pulses for long durations between recharges.

A basic square wave may be employed where the voltage rises rapidly to a set amplitude and remains constant for a defined pulse width (e.g., microseconds to milliseconds). These pulses are well-studied for bacterial cell membrane disruption, offering reliable electroporation results. Biphasic pulses switch polarity midway through the pulse, potentially reducing electrode material degradation and delivering more uniform electroporation to bacterial cells. In some examples, the system may alternate positive and negative pulses in rapid succession to optimize microbial kill rates. Exponential decay pulses may be used when specific voltage modulation is required to avoid thermal damage. By tapering the voltage, the system can maintain sufficiently high fields initially, then gradually reduce the energy input, mitigating potential adverse effects on healthy tissue. Short bursts of high-frequency pulses (e.g., 1-5 kHz) can be beneficial for deep-tissue electroporation or when targeting multi-layered biofilms. This approach may rapidly deliver the necessary electric field while keeping total energy exposure lower than a single prolonged pulse.

Certain examples use electroporation not only for sterilization but to enhance local drug uptake. For example, an antibiotic solution may be infused through the catheter shortly before pulsed fields are activated, temporarily increasing bacterial cell permeability and boosting antimicrobial effectiveness. A hip or knee prosthesis or implant might integrate an internal sensor array that detects micro-movement or changes in local tissue impedance indicative of infection. Intermittent electroporation pulses can disrupt biofilm formation at the bone-implant interface. Patients at high risk for chronic infection (e.g., immunocompromised individuals) particularly benefit from such prophylaxis. Another example includes a multi-lumen catheter: one lumen dedicated to fluid infusion, another housing electrical leads and sensors, and a third delivering specialized treatments or flush solutions. The electroporation pulses can be targeted through one lumen while normal fluid administration continues through the other lumens uninterrupted. In certain examples, the control unit or software algorithm schedules regular sterilization cycles at programmable intervals (e.g., daily, weekly). The device logs each activation event, voltage level, and duration, allowing clinicians to analyze efficacy and adjust parameters over time.

In one example, an electroporation system utilizes nanosecond pulsed electric fields (nsPEFs) to induce intracellular effects distinct from conventional electroporation techniques. The device delivers ultra-short pulses of less than 100 nanoseconds, disrupting bacterial metabolic processes and organelles while minimizing thermal damage to surrounding tissues. By operating at nanosecond timescales, the system achieves a unique bacterial inactivation mechanism compared to existing pulsed field ablation (PFA) approaches. The nsPEF system may also be configured with adjustable pulse amplitudes and repetition rates, allowing customization for different bacterial strains and clinical scenarios. Advanced models could incorporate feedback control using impedance monitoring to adjust pulse parameters dynamically, ensuring optimal bacterial disruption while preserving host tissue integrity.

In another example, the electroporation system applies low-intensity alternating current (AC) fields at optimized frequencies to selectively disrupt bacterial cells. Unlike pulsed DC fields, AC electroporation does not require high-voltage transients, reducing power consumption and extending device longevity. This approach allows for continuous, low-energy bacterial suppression without relying on high-intensity pulsed fields. Additionally, modulating the frequency of AC fields can enhance selectivity for bacterial disruption while preserving host tissue integrity. Advanced implementations could incorporate frequency sweeps to optimize cell disruption across a spectrum of microbial species while preventing adaptive resistance.

Alternatively, in another example, capacitive coupling is employed instead of direct electrode contact with body fluids. A dielectric layer between the electrodes and the surrounding medium enables charge transfer via capacitive effects, allowing electroporation to occur without electrode degradation. This method provides a non-invasive mechanism of field application that minimizes unwanted electrochemical reactions. Capacitive coupling can also be integrated with an adaptive feedback system to adjust field strength dynamically based on tissue impedance. Furthermore, advanced materials, such as biocompatible polymer coatings with embedded conductive nanoparticles, could be utilized to enhance charge transfer efficiency.

In one example, a catheter-based electroporation system features external electrode arrays positioned on the patient's skin near the catheter insertion site. These arrays generate electric fields that propagate through tissue and catheter components, allowing non-invasive electroporation without requiring internal catheter electrodes. This design enhances compatibility with existing catheters while avoiding the need for embedded leads. The external electrode system may also incorporate impedance monitoring to adjust field strength and optimize bacterial elimination. Additionally, electrode configurations could be dynamically reconfigured based on tissue conductivity measurements, optimizing treatment delivery.

In another example, the system incorporates interdigitated microelectrodes (IDMEs) on the catheter's outer surface. These patterned microelectrodes create non-uniform electric fields that disrupt bacterial biofilms more effectively than traditional ring electrodes. The localized field distribution alters bacterial adhesion properties, enhancing biofilm elimination without increasing energy demands. The IDMEs may be arranged in a reconfigurable pattern to allow targeted delivery of electroporation pulses to specific regions. Additionally, variable electrode spacing could be used to manipulate local field intensities and optimize bacterial cell wall disruption.

Alternatively, in yet another example, nanoelectrode arrays composed of conductive nanomaterials such as graphene or carbon nanotubes are integrated into the catheter surface. These nanoscale electrodes generate highly localized electric fields at the bacterial scale, achieving bacterial inactivation while using lower power levels compared to macroelectrode systems. The nanoelectrode arrays can be designed to work in conjunction with an antimicrobial surface coating to enhance sterility and device longevity. Advanced designs may integrate temperature-sensitive nanomaterials to trigger additional antimicrobial effects when electroporation is applied.

In one example, a standalone electroporation catheter system is developed, featuring an independent control module that enables electroporation without requiring a connection to an implantable cardiac device. The control module can be positioned externally or within the catheter itself, allowing flexible treatment application. This configuration allows the system to be used in various medical settings without requiring integration with existing pacemaker or defibrillator platforms. The control module could be integrated with AI-based diagnostics to recommend optimal electroporation parameters based on real-time sensor feedback.

In another example, the system employs a wearable external power source in the form of a small, clip-on or patch-based unit. This wearable power unit activates electroporation sessions on demand, eliminating the need for an implanted pulse generator while maintaining portability. The device may feature wireless communication capabilities, enabling remote activation by healthcare providers. Future designs could incorporate energy-harvesting technologies to extend battery life and minimize recharging requirements.

Alternatively, wireless power transfer (inductive coupling) is used to energize the catheter's electrodes. This system relies on external coils to generate electromagnetic fields that wirelessly transmit energy to embedded electrodes, providing electroporation therapy without physical wiring. This technique allows for power delivery without direct contact, reducing the risk of infection at connection points. Optimized coil geometries and frequency tuning could improve energy efficiency and treatment efficacy.

In one example, the electroporation system is designed to target fungal infections such as Candida within urinary catheters. Unlike bacterial biofilm-focused devices, this system operates with parameters optimized for fungal cell wall disruption, distinguishing it from traditional electroporation applications. The fungal-targeting system may include adjustable pulse widths tailored for different fungal species. Additionally, selective frequency tuning may further enhance antifungal effects.

In another example, the system enhances antibiotic penetration into bacterial cells instead of directly lysing them. By applying controlled pulses, bacterial membranes become temporarily permeabilized, increasing drug uptake and reducing antibiotic resistance development. This approach allows lower doses of antibiotics to be more effective, reducing potential side effects. Additionally, time-gated electroporation pulses could optimize drug uptake efficiency while minimizing bacterial repair mechanisms.

Alternatively, in yet another example, immune cell stimulation is achieved using low-intensity pulsed electric fields. This technique activates immune responses near the catheter, enhancing the body's natural defense mechanisms against infections without directly targeting bacterial cells. The immune-modulating pulses can be tuned to stimulate specific cytokine release patterns, promoting enhanced localized immunity. Advanced configurations could integrate real-time immune response monitoring to adjust field parameters dynamically.

Indwelling vascular catheters and other medical implants frequently trigger immune and inflammatory responses, leading to device failure over time. The present disclosure provides systems and methods that leverage electroporation or pulsed field ablation (PFA) to mitigate inflammation, inhibit fibrosis, and prevent catheter dysfunction.

Upon catheter implantation, the body initiates a foreign body response (FBR), leading to macrophage activation, fibroblast proliferation, and extracellular matrix deposition around the catheter surface. Over time, this fibrotic encapsulation may cause vessel narrowing, catheter occlusion, and reduced blood flow.

To address this, electroporation pulses may be delivered in a controlled manner to selectively disrupt excessive fibroblast activity while preserving endothelial integrity. The system may: deliver low-energy electroporation pulses at predefined intervals to modulate fibroblast proliferation; synchronize electroporation with impedance feedback monitoring, allowing adaptive pulse delivery to areas of high fibrotic tissue density; and/or utilize catheter-embedded electrodes for localized pulsed field treatment, reducing fibrosis around dialysis catheters, peripherally inserted central catheters (PICCs), or vascular grafts.

Biofilm formation on catheter surfaces acts as a persistent trigger for chronic inflammation, neutrophil activation, and cytokine release (e.g., IL-6, TNF-α, IL-1β). These inflammatory mediators further recruit immune cells, trigger platelet aggregation, and increase the risk of thrombosis, leading to catheter blockage.

The disclosed system utilizes electroporation electrodes positioned along, within, or around the catheter to disrupt biofilms and modulate immune signaling. Short, controlled pulses can target bacterial biofilms, breaking down microbial colonies that trigger chronic inflammation. Real-time impedance monitoring detects early-stage biofilm formation, enabling preemptive electroporation pulses before full biofilm maturation. Electroporation-enhanced drug delivery can increase the permeability of bacterial membranes, improving the efficacy of antibiotic lock solutions used in catheter maintenance.

Catheters can induce platelet activation, endothelial damage, and fibrin sheath formation, which promote thrombosis and catheter occlusion. This is particularly problematic in dialysis access devices, peritoneal dialysis catheters, and long-term venous access systems. The present system addresses thrombosis risk by: applying electroporation pulses at the catheter tip or insertion site to reduce platelet aggregation and inhibit thrombus formation; integrating electroporation with an anticoagulant coating (e.g., heparin-infused catheters) to synergistically prevent clot formation; utilizing real-time impedance feedback to adjust electroporation intensity based on early clot detection, preventing complete catheter occlusion.

In some examples, the system may include adaptive control algorithms that synchronize electroporation pulses with drug delivery. For instance: Dexamethasone or other anti-inflammatory agents may be delivered through the catheter lumen, with electroporation pulses used to increase local tissue permeability and enhance drug diffusion. Electroporation-driven bacterial cell permeabilization can be used in combination with antibiotic lock therapy, improving bacterial eradication in infected catheters.

By incorporating electroporation-based immune modulation, biofilm disruption, and thrombosis prevention, the disclosed systems provide an innovative approach to prolong catheter function, reduce infection rates, and mitigate immune-mediated catheter failure.

The system may implement multi-phase electroporation therapy to modulate the immune response over time rather than relying on a single pulse application. In the early phase, spanning 0-48 hours post-implantation, low-energy pulses may be administered to prevent acute macrophage activation and fibroblast proliferation. This stage is critical in mitigating an aggressive foreign body response. In the mid-phase, occurring between 2-14 days post-implantation, higher-intensity pulses may target early-stage fibrosis and biofilm adhesion, reducing the likelihood of persistent inflammatory buildup. In the chronic phase, beyond 14 days, sustained pulses may be delivered to inhibit long-term fibrotic encapsulation and maintain catheter patency. The system may adapt pulse parameters based on real-time impedance sensing, which can detect the progression of fibrotic tissue growth, bacterial colonization, or thrombus formation.

Fibrotic development around implanted catheters is often non-uniform, with specific regions experiencing higher fibrotic deposition. To address this, the system may selectively activate different electrode segments along the catheter to target areas with excessive fibrotic response. Electrode arrays arranged in a segmented configuration may allow for differential energy delivery based on real-time feedback from bioimpedance sensors. By precisely controlling energy distribution, the system can mitigate localized fibrotic buildup without affecting surrounding healthy tissue.

While electroporation effectively disrupts biofilms, biofilm fragments may still adhere to catheter surfaces, posing a risk of recolonization. To counter this, the system may integrate low-frequency ultrasonic vibration or microfluidic shear forces in conjunction with electroporation. These mechanical forces can dislodge biofilm fragments after disruption by pulsed fields, preventing bacterial adhesion and recolonization. The synergistic approach enhances biofilm clearance while minimizing the risk of persistent bacterial growth.

Endothelial cells are essential in modulating vascular inflammation and maintaining blood flow. The system may be designed to deliver biphasic or asymmetric electroporation pulses optimized to selectively target pro-inflammatory immune cells while preserving endothelial integrity. Additionally, the system may minimize disruption of nitric oxide signaling, reducing unintended vasoconstriction or endothelial dysfunction. In some cases, controlled electroporation parameters may stimulate the release of vascular endothelial growth factor (VEGF), supporting endothelial repair and regeneration.

Beyond biofilm prevention, electroporation may also serve as an anti-thrombogenic mechanism. The system may modulate coagulation factor activity through pulsed field applications, reducing fibrinogen conversion and preventing early clot formation. Additionally, electroporation may inhibit platelet aggregation near the catheter tip and regulate tissue factor expression, minimizing pro-thrombotic signaling. These targeted applications can significantly reduce the risk of catheter-related thrombosis while preserving vascular function.

Implantable electroporation-enabled catheters may be equipped with remote adjustability features, allowing clinicians to modify electroporation parameters based on patient-specific inflammation or biofilm progression. The system may support on-demand electroporation pulses triggered by infection biomarkers detected in real-time. Additionally, waveform profiles may be modified to accommodate different stages of the foreign body response, ensuring continuous protection against infection and fibrotic encapsulation.

Drug-eluting catheters traditionally release anti-inflammatory agents, such as dexamethasone or sirolimus, through passive diffusion. Electroporation may enhance this process by actively accelerating drug release at strategic time points. For instance, short, high-voltage pulses immediately post-implantation may boost early anti-inflammatory drug diffusion, reducing acute immune responses. Periodic low-energy pulses may be applied to prolong sustained drug release over weeks to months. Additionally, region-specific electroporation can trigger localized drug diffusion, particularly in areas where biofilm formation or fibrosis is most pronounced. This targeted drug release strategy enhances therapeutic efficacy while reducing the risk of systemic side effects.

In one example, electroporation is activated via manual control, allowing healthcare providers or patients to trigger the therapy as needed. This approach avoids automated sensor-based activation, reducing the complexity of real-time feedback mechanisms. The manual control interface may include programmable settings for pulse intensity and duration. Additionally, smart wearable interfaces could provide real-time treatment tracking and patient feedback.

In another example, the system implements pre-treatment electroporation, wherein catheters are sterilized before insertion. This method ensures a biofilm-resistant surface from the outset, preventing bacterial colonization rather than treating existing infections. Pre-treatment can be applied as a rapid, high-energy pulse session just prior to use in clinical settings. Enhanced sterilization protocols may include automated surface charge rebalancing to prevent bacterial adhesion.

In one example, the electroporation device integrates ultraviolet (UV) or blue light sterilization alongside electric field application. The dual-modality system leverages light-induced antimicrobial effects in combination with electroporation to achieve superior bacterial elimination. UV light can be emitted in conjunction with electroporation pulses for enhanced disinfection. Advanced UV timing algorithms may further optimize bacterial elimination.

In another example, low-frequency ultrasound is combined with electroporation to disrupt bacterial biofilms mechanically before applying electrical pulses. This synergistic approach improves bacterial eradication while using lower electroporation intensities. The ultrasound waves may be pulsed in synchronization with electroporation for optimal biofilm penetration.

Ensuring that electroporation effectively targets bacterial cells while preserving the integrity of surrounding tissues is a critical design consideration. The system incorporates multiple safety mechanisms to regulate the intensity, duration, and frequency of electroporation pulses. Advanced control algorithms dynamically adjust electroporation parameters based on real-time sensor feedback, minimizing the risk of unintended cellular disruption.

Some examples include the integration of impedance-based monitoring to differentiate bacterial biofilms from host tissues. By continuously measuring electrical resistance across electrodes, the system can determine whether electroporation pulses are appropriately applied to the intended target. If impedance values suggest proximity to delicate tissues, the pulse parameters are automatically adjusted or deactivated to prevent unwanted cell damage.

Additionally, waveform optimization may be employed to minimize thermal effects. Traditional electroporation pulses can generate localized heating, which may compromise tissue viability. To counteract this, the system utilizes carefully tuned biphasic, asymmetric, and exponential decay waveforms, which deliver controlled energy while reducing heat accumulation. These waveforms ensure effective bacterial eradication while maintaining a safe thermal profile for surrounding tissues.

To further enhance compatibility, the electrodes may be coated with biocompatible insulating layers that limit direct contact with non-target tissues. These coatings provide a controlled electrical field distribution, ensuring that electroporation occurs only within the intended microbial zones. Moreover, programmable electroporation cycles allow clinicians to tailor treatment parameters to specific infection sites, ensuring precise and patient-specific therapy.

For fully implanted devices, some examples include an inductive wireless charging system allows for periodic recharging without requiring invasive interventions. The system utilizes a low-profile, transdermal energy transfer module, enabling the external charging unit to deliver power efficiently while minimizing heat buildup. This feature extends battery life and allows for flexible, on-demand electroporation cycles.

Additionally, energy-harvesting technologies may be integrated to passively collect power from physiological movements, such as cardiac contractions in pacemaker-integrated systems. This secondary power source reduces the reliance on frequent recharging and extends device longevity.

For temporary or percutaneous applications, the device may incorporate removable power tethers that allow direct connection to an external power supply. These tethers provide high-power electroporation pulses when needed, such as during intensive infection treatments, while allowing detachment once therapy is complete.

To ensure reliability, the power management unit may include smart energy regulation circuits that dynamically adjust voltage and current output based on real-time impedance feedback. This prevents excessive power delivery, mitigating risks of thermal injury or unintended stimulation of surrounding tissues.

The system may also feature fail-safe mechanisms that prevent unintended electroporation activation due to electrical fluctuations, software malfunctions, or improper device handling. These safety protocols ensure that power delivery remains stable and precise throughout the device's operational lifespan. By integrating efficient power management with safety-driven tissue compatibility measures, the electroporation system ensures optimal infection control without compromising patient safety or device longevity.

Continuous or on-demand electroporation significantly diminishes microbial colonization on surfaces of indwelling devices, including catheters, ports, or implants. By leveraging external power delivery or small on-board capacitors, the implant can remain compact. This is particularly valuable for patients needing multiple implanted devices or those requiring minimal invasive hardware. The inventive concepts described here can be adapted to numerous clinical contexts, from orthopedics and cardiology to dialysis catheters and neurostimulation leads. Electroporation modules can be configured to meet different pulse requirements, anatomical constraints, and infection prevalence rates. Sensor data, such as temperature and impedance, helps maintain safe operating thresholds, ensuring that healthy tissue is not adversely affected by repeated high-voltage pulses. Automatic shutoffs or pulse modulation algorithms can be implemented to prevent thermal or mechanical damage.

It should be recognized that the foregoing examples represent merely a subset of possible implementations. Those skilled in the art will appreciate that various modifications, materials, and design choices may be employed to tailor the devices for specific clinical uses or patient profiles. The systems, methods, and devices herein described serve as a foundation for implementing electroporation-based infection prevention and treatment strategies across a diverse range of medical applications.

It will be appreciated that elements or components shown with any example herein are exemplary for the specific example and may be used on or in combination with other examples disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A system for mitigating infection, comprising:
an elongate member configured for insertion into a biological conduit, the elongate member having a proximal end adapted for connection to a fluid exchange system and a distal end sized to be positioned within the biological conduit;
at least one electrode positioned at least one of along, within, or around the elongate member; and
a control unit configured to generate and apply pulsed electric fields via the at least one electrode such that the electric fields are applied to at least one of the elongate member, surrounding biological structures, or a region within or around the biological conduit.

2. The system of claim 1, wherein the control unit is configured to apply the pulsed electric fields via the at least one electrode at a field strength between 500 V/cm and 4000 V/cm, with pulse durations between nanoseconds to milliseconds.

3. The system of claim 2, wherein the control unit is configured to deliver the pulsed electric fields using at least one of a monophasic, biphasic, exponential decay, or sinusoidal waveform, wherein the control unit is configured to adjust waveform parameters, including frequency and duty cycle of the waveform, to facilitate bacterial eradication while minimizing effects on surrounding human tissue.

4. The system of claim 1, wherein the at least one electrode comprises a plurality of ring electrodes spaced along the length of the elongate member, the ring electrodes configured to generate a uniform electric field within the biological conduit to enable localized or full-length electroporation treatment.

5. The system of claim 1, wherein the at least one electrode comprises at least one of a flexible conductive polymer, metallic deposition, or nanostructured coating, the electrode applied to at least one of the inner or outer surface of the elongate member to facilitate continuous or segmental electroporation.

6. The system of claim 1, wherein the at least one electrode comprises at least one of an interdigitated electrode array, a mesh-based electrode structure, or a helical electrode design, the electrode configured to enhance spatial uniformity of the applied electric field.

7. The system of claim 1, wherein the control unit is configured to modulate at least one of pulse intensity, duration, and frequency based on at least one of real-time sensor feedback, impedance detection, bacterial load, or biofilm formation to enable adaptive electroporation therapy.

8. The system of claim 7, wherein the control unit is configured to interface with at least one of a dialysis machine, an infusion pump, or a patient monitoring system, the interface enabling remote adjustment of electroporation parameters based on real-time physiological data.

9. The system of claim 1, further comprising at least one of a deployable sheath or an expandable electrode assembly, the sheath or assembly configured to conform to the biological conduit upon insertion and to optionally retract after electroporation treatment.

10. The system of claim 1, further comprising an expandable electroporation mesh positioned at the distal end of the elongate member, the mesh configured to one or more of: (i) deploy within a biological conduit or vascular lumen to conform to the surrounding anatomy; (ii) apply pulsed electric fields to disrupt bacterial biofilms and bloodstream infections; and (iii) retract, collapse, or dissolve after treatment to allow for temporary or semi-permanent applications.

11. The system of claim 1, wherein the elongate member is configured for at least one of dialysis, peritoneal therapy, vascular access, or urinary catheterization, the at least one electrode positioned on the elongate member to apply pulsed electric fields for bacterial disruption and biofilm prevention.

12. The system of claim 1, further comprising an external electroporation interface, the interface comprising at least one of a flexible adhesive patch, a detachable cap, and a conductive hydrogel layer configured to apply pulsed electric fields to a dialysis port or vascular access site.

13. The system of claim 7, wherein the control unit is configured to be powered by at least one of an implantable or external power source, with power delivery provided via at least one of inductive coupling, direct connection, battery operation, or energy harvesting from fluid flow.

14. The system of claim 13, wherein the control unit comprises a rechargeable capacitor bank or wireless energy transfer system, enabling periodic electroporation treatment without continuous power consumption.

15. A method for mitigating infection, comprising:
positioning an elongate member within a biological conduit, the elongate member having at least one electrode;
generating pulsed electric fields via the at least one electrode; and
applying the pulsed electric fields to at least one of the elongate member and surrounding biological structures to disrupt microbial activity and mitigate infection.

16-22. (canceled)

23. A system for mitigating immune response to an implanted medical device, comprising:
an elongate member configured for insertion into a biological conduit, the elongate member having a proximal end adapted for connection to a fluid exchange system and a distal end sized to be positioned within the biological conduit;
at least one electrode positioned at least one of along, within, or around the elongate member; and
a control unit configured to generate and apply pulsed electric fields via the at least one electrode such that the electric fields are applied to at least one of the elongate member and surrounding biological structures to modulate immune activity.

24. The system of claim 23, wherein the control unit is configured to deliver the pulsed electric fields to reduce activation of at least one of macrophages, T cells, or dendritic cells in proximity to the implanted medical device.

25. The system of claim 23, wherein the control unit is configured to deliver the pulsed electric fields to modulate cytokine release at the implantation site to reduce inflammatory response.

26. The system of claim 23, wherein the control unit is configured to deliver the pulsed electric fields to alter permeability of immune cells to facilitate delivery of immunomodulatory agents, wherein the system further comprises a drug-eluting component configured to enhance uptake of at least one of an anti-inflammatory or immunosuppressive agent at the implantation site.

27. The system of claim 23, further comprising a biodegradable sheath positioned around the elongate member, the sheath being configured to deliver pulsed electric fields to mitigate early-stage immune rejection and dissolve over time.

28-36. (canceled)
